(19) United States
(12) Patent Application Publication
(10) Pub. No.: US 2018/0311334 A1
(43) Pub. Date: Nov. 1, 2018

(54) Compositions and Methods for Inducing an Immune Response

(71) Applicant: Yale University, New Haven, CT (US)

(72) Inventors: Samir Gautam, New Haven, CT (US); Taehan Kim, New Haven, CT (US); David Spiegel, New Haven, CT (US)

(21) Appl. No.: 15/964,322

(22) Filed: Apr. 27, 2018

Related U.S. Application Data
(60) Provisional application No. 62/492,129, filed on Apr. 29, 2017.

(51) Int. Cl. A61K 39/085; A61K 39/39; A61P 31/04

(52) U.S. Cl. CPC A61K 39/085; A61K 2039/572; A61P 31/04; A61K 39/39

(57) ABSTRACT

The invention provides a modified cell having an immunomodulatory molecule bound to the cell surface. In some aspects, the invention provides compositions and methods for the treatment and prevention of a microbial infection and microbial infection-related diseases and disorders. The present invention also provides methods of inducing an immune response against a pathogen.

### CROSS REFERENCE TO RELATED APPLICATIONS

This application claims priority to U.S. Provisional Application No. 62/492,129, filed Apr. 29, 2017 which is hereby incorporated by reference herein in its entirety.

### STATEMENT REGARDING FEDERALLY SPONSORED RESEARCH OR DEVELOPMENT

This invention was made with government support under OD002913, GM100283 and GM007205 awarded by National Institutes of Health. The government has certain rights in the invention.

### BACKGROUND OF THE INVENTION

Researchers have eagerly sought to develop a Staphylococcus aureus vaccine for over half a century, but with limited success. Evidence has recently emerged that vaccine polyantigenicity and T cell-promoting properties may be essential for vaccine efficacy against S. aureus, but no vaccine has yet been developed that meet these two criteria.

Accordingly, there exists a need for improved compositions and methods for the prevention and treatment of S. aureus and other bacterial infections. The present invention meets this need.

### SUMMARY OF THE INVENTION

In one aspect, the present invention provides a cell comprising an immunomodulatory molecule bound to the cell surface. In one embodiment, the cell is a bacterial cell or a fungal cell. In one embodiment, the bacterial cell is an inactivated bacterial cell. In one embodiment, the bacterial cell is Staphylococcus sp. cell. In one embodiment, the bacterial cell is an inactivated bacterial cell. In one embodiment, the Staphylococcus sp. cell is Staphylococcus aureus cell.

In one embodiment, the immunomodulatory molecule comprises an adjuvant group, a linker group and a targeting group.

In one embodiment, the targeting group selected from the group consisting of a group capable of participating in a click-chemistry reaction and a group capable of participating in a sortase-mediated labeling reaction. In the group capable of participating in a click-chemistry reaction comprises a group selected from the group consisting of a terminal alkyne, a terminal azide, and a cyclooctyne group. In one embodiment, the group capable of participating in a sortase-mediated labeling reaction comprises a sortase targeting peptide. In one embodiment, the sortase targeting peptide comprises the amino acid sequence LPETG (SEQ ID NO:1).

In one embodiment, the linker group bridges the adjuvant group and the targeting group. In one embodiment, the linker comprises a group selected from the group consisting of a disulfide linking group, a thioester group, and a polyethylene glycol (PEG) group.

In one embodiment, the adjuvant comprises a group selected from the group consisting of a imidazoquinoline and a imidazoquinoline derivative. In one embodiment, the immunomodulatory molecule is:

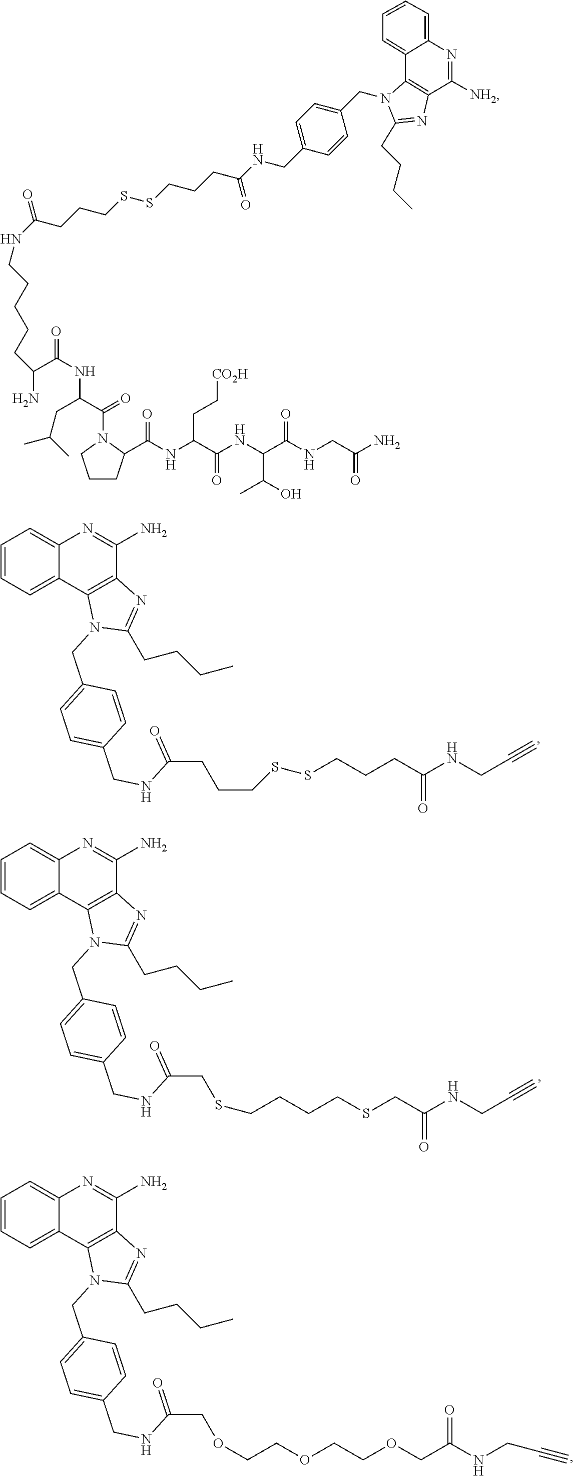

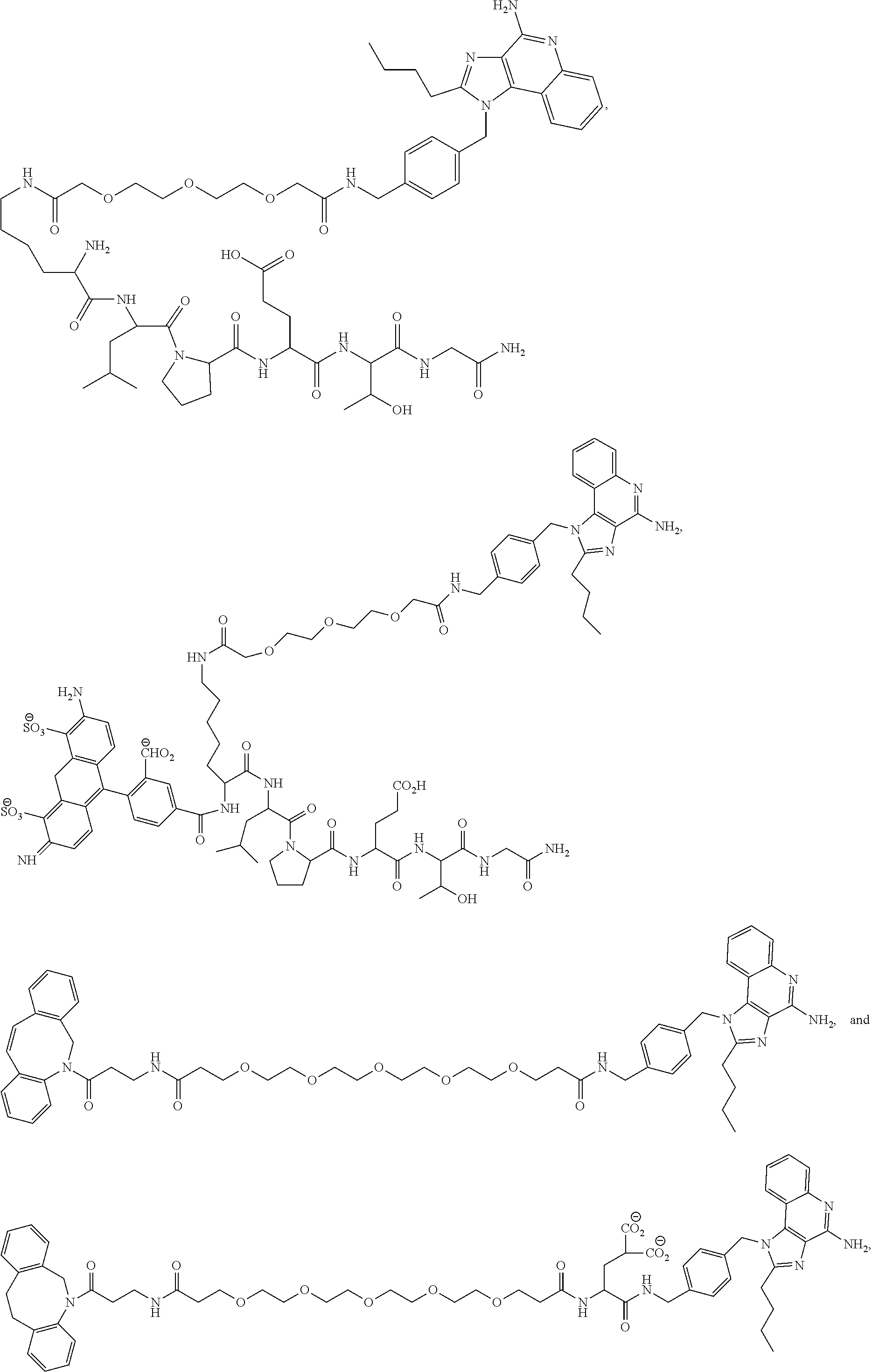

or any derivative thereof.

In one embodiment, the invention provides an immunological composition comprising a cell comprising an immunomodulatory molecule bound to the cell surface.

In one embodiment, the invention provides a method for treating or preventing an infection in a subject. In one embodiment, the method comprises administering to the subject a composition comprising a cell comprising an immunomodulatory molecule bound to the cell surface. In one embodiment, the infection is an infection of a pathogenic bacterium. In one embodiment, the infection is a Staphylococcus aureus infection.

In one embodiment, the invention provides a method of inducing an immune response in a subject. In one embodiment, the method comprises administering to the subject a composition comprising a cell comprising an immunomodulatory molecule bound to the cell surface.

In one embodiment, the invention provides a method for linking a molecule to a cell surface. In one embodiment, the cell comprises a free amine on the cell surface. In one embodiment, the method comprises attaching a first moiety capable of participating in a click chemistry reaction to the free amine on the surface of the cell; and incubating the cell with a mixture comprising a molecule comprising a second moiety capable of participating in a click chemistry reaction. In one embodiment, the mixture further comprises copper

In one embodiment, the step attaching a first moiety capable of participating in a click chemistry reaction to a free amine on the surface of the cell comprises attaching an azide to a free amine on the surface of the cell.

In one embodiment, the second moiety capable of participating in a click chemistry reaction is selected from the group consisting of a terminal alkyne and a cyclooctyne.

### BRIEF DESCRIPTION OF THE DRAWINGS

The foregoing summary, as well as the following detailed description of exemplary embodiments of the invention, will be better understood when read in conjunction with the appended drawings. It should be understood, however, that the invention is not limited to the precise arrangements and instrumentalities of the embodiments shown in the drawings.

FIG. 1 is a schematic representing the rationale of the vaccine design. Attachment of adjuvant to S. aureus leads to activation of dendritic cells via ligation of pathogen recognition receptors, with consequent elicitation of CD4⁺ T-cell responses, which protect against future staphylococcal infection.

FIG. 2 is a detailed schematic overview of the vaccine design. Adjuvants chemically conjugated to the surface of S. aureus bind to pathogen recognition receptors, leading to activation of dendritic cells (DCs). DCs then stimulate CD4⁺ T cells via three signals: TCR recognition of staphylococcal antigens presented on MHCII; ligation of surface receptors on T cells with co-stimulatory proteins; and secretion of cytokines that potentiate and tune specific T cell responses. S. aureus-specific T cells, in turn, provide protection against future infection. Abbreviations: MHCII, major histocompatibility complex class II; PAMP, pathogen associated molecular pattern; PRR, pathogen recognition receptor; TCR, T cell receptor.

FIG. 3, comprising FIG. 3A through FIG. 3C, depicts the structure of Sortase-targeted-Disulfide-Xyliquimod (SDX). FIG. 3A depicts the prototype compound for these studies, SDX. SDX comprises a sortase targeting peptide motif, LPETG (SEQ ID NO:1), which is connected to xyliquimod (an imidazoquinoline TLR7 agonist) through an additional lysine residue and a disulfide linker region. FIG. 3B depicts the structure of xyliquimod-thiol, the reductive cleavage product of SDX. FIG. 3C depicts the TLR7 activity of xyliquimod-thiol. mTLR7-positive and -negative NFκB-Luc expressing HEK cells were treated with soluble xyliquimod-thiol. After 22 hours, cells were lysed and assayed for luciferase activity. Results are expressed as fold increase in luminescence over mTLR7-negative control levels. Experiments performed in technical triplicate; error bars represent SEM.

FIG. 4, comprising FIG. 4A through FIG. 4C, depicts experimental results demonstrating that xyliquimod stimulates immune responses when conjugated to S. aureus through either cleavable or non-cleavable linkers. FIG. 4A depicts a schematic of addition of azide moieties onto the surface of S. aureus. WT S. aureus Newman was treated with azidoacetic acid N-hydroxysuccinimide ester (AAA-NHS) to install azide moieties on the cell surface. Terminal alkyne-equipped imidazoquinoline derivatives were then ligated to azide handles via copper-mediated click chemistry. FIG. 4B depicts the structures of alkyne-functionalized xyliquimod derivatives, PDX and PTX. FIG. 4C depicts the fold increase in luminance of S. aureus with PDX or PTX over luminescence induced by untreated heat-killed S. aureus alone (1 MOI). NFκB-sLuc expressing RAW macrophages were treated with soluble adjuvants (1 μM), adjuvant conjugated to heat-killed S. aureus (1 MOI), or adjuvant non-specifically adsorbed to heat-killed S. aureus (1 MOI). After 15 hours, the supernatant was assayed for luciferase activity.

FIG. 5, comprising FIG. 5A through FIG. 5D, depicts experimental results demonstrating that a PEGS-linked alkynyl derivative of xyliquimod (PPX) elicits TLR7-dependent immune responses when conjugated to S. aureus. FIG. 5A depicts the structure of the PEGS-linked alkynyl xyliquimod derivative, PPX. FIG. 5B depicts the comparative activity of soluble alkynyl xyliquimod derivatives in RAW cells. NFκB-sLuc expressing RAW macrophages were treated with varying concentrations of soluble adjuvants for 16 hours and the supernatant was assayed for luciferase activity. Results are expressed as fold increase over unstimulated macrophages FIG. 5C depicts the activity of PPX-conjugated S. aureus in RAW cells. NFκB-sLuc expressing RAW macrophages were treated with PPX conjugated or PPX-adsorbed heat-killed S. aureus at various MOI. After 10 hours, the supernatant was assayed for luciferase activity. FIG. 5D depicts the activity of PPX-conjugated S. aureus in mTLR7-HEK cells. mTLR7-positive and -negative NFκB-Luc expressing HEK cells were treated with PPX-conjugated or PPX-adsorbed heat-killed S. aureus (20 MOI). After 22 hours, cells were lysed and assayed for luciferase activity. Experiments performed in technical triplicate; error bars represent SEM.

FIG. 6 depicts the structure of Sortase-targeted-PEG3-Xyliquimod (SPX). The second-generation compound used in these studies, SPX, comprises a sortase targeting peptide motif, LPETG (SEQ ID NO:1), connected to xyliquimod through an additional lysine residue and a PEG3 linker region.

FIG. 7, comprising FIG. 7A through FIG. 7D, depicts experimental results demonstrating that the addition of SPX augments immune responses to S. aureus, but in a sortase-independent manner. FIG. 7A depicts the activity of SPX-conjugated S. aureus in RAW cells. NFκB-sLuc expressing RAW macrophages were stimulated with SPX-treated heat-killed S. aureus at various MOI. After 4 hours, the supernatant was assayed for luciferase activity. FIG. 7B depicts the effects of sortase expression on activity of SPX-conjugated S. aureus in RAW cells. NFκB-sLuc expressing RAW macrophages were stimulated with WT or srtA-S. aureus that had been treated with SPX and then heat-killed (20 MOI). After 4 hours, the supernatant was assayed for luciferase activity. FIG. 7C depicts the structure of EPX: a PEG3-linked xyliquimod derivative attached to the scrambled control peptide, EGTLP. FIG. 7D depicts the activity of EPX-treated S. aureus in RAW cells. NFκB-sLuc expressing RAW macrophages were stimulated with SPX- or EPX-treated heat-killed S. aureus (10 MOI). After 8 hours, the supernatant was assayed for luciferase activity. Experiments performed in technical triplicate; error bars represent SEM.

FIG. 8, comprising FIG. 8 through FIG. 8D, depicts experimental results demonstrating that the fluorescent and polar SPX variant, ASPX, is incorporated into the cell wall of S. aureus by sortase and is active in soluble form, but does not augment immune responses when conjugated to S. aureus. FIG. 8A depicts the structure of ASPX: a PEG3-linked xyliquimod derivative attached to Alexa 488 and the sortase targeting peptide, LPETG (SEQ ID NO:1). FIG. 8B depicts the effects of sortase expression on incorporation of ASPX. WT and srtA-S. aureus were incubated with ASPX for 16 hours. Labeling was assessed by measuring total cellular FL-1 fluorescence on a flow cytometer. FIG. 8C depicts the activity of soluble ASPX in RAW cells. NFκB-sLuc expressing RAW macrophages were treated with varying concentrations of soluble ASPX and SPX for 4 hours and the supernatant was assayed for luciferase activity. FIG. 8D depicts the activity of ASPX-treated S. aureus in RAW cells. NFκB-sLuc expressing RAW macrophages were stimulated with either WT or srtA-S. aureus that had been treated with ASPX and then heat-killed (10 MOI). After 4 hours, the supernatant was assayed for luciferase activity. Luciferase assays performed in technical triplicate; error bars represent SEM.

FIG. 9 depicts primary cell experiments confirming that SPX augments immune responses to S. aureus in a sortase-independent manner, and that ASPX fails to improve immunogenicity at all. WT and TLR7KO murine bone marrow-derived macrophages were stimulated for 12 hours with WT or srtA-S. aureus that had been labeled overnight with SPX, ASPX, or no peptide and then heat-killed (20 MOI). Stimulation with 1 μM xyliquimod was included as a control. mRNA expression of IL-1β was assessed to determine the magnitude of immune responses; results are expressed as fold increase over unstimulated macrophages. Experiment performed in technical triplicate; error bars represent SEM.

FIG. 10 depicts experimental results demonstrating that PPX conjugation dramatically improves the immunogenicity of S. aureus through activation of TLR7. WT and TLR7KO murine bone marrow-derived macrophages were stimulated for 12 hours with heat-killed S. aureus conjugated to PPX (20 MOI). Untreated S. aureus, PPX-adsorbed S. aureus, and S. aureus co-administered with 1 nM soluble PPX (equimolar to that conjugated to the bacteria) were included as controls. mRNA expression of IL-1β was assessed to determine the magnitude of immune responses; results are expressed as fold increase over unstimulated macrophages. Experiment performed in technical triplicate; error bars represent SEM.

FIG. 11 depicts experimental results demonstrating that PPX conjugation to S. aureus elicits a TLR7-dependent type I interferon response. WT and TLR7KO murine bone marrow-derived macrophages were stimulated for 12 hours with heat-killed S. aureus conjugated to PPX (20 MOI). Untreated S. aureus, PPX-adsorbed S. aureus, and S. aureus co-administered with 1 nM soluble PPX (equimolar to that conjugated to the bacteria) were included as controls. mRNA expression of IFNβ was assessed by qPCR; results are expressed as fold increase over unstimulated macrophages. Experiment performed in technical triplicate; error bars represent SEM.

FIG. 12 depicts experimental results demonstrating that PPX conjugation stimulates expression of pro-inflammatory and Th1-promoting cytokines via TLR7. WT and TLR7KO murine bone marrow-derived macrophages were stimulated for various times with heat-killed S. aureus conjugated to PPX (20 MOI). 1 μM soluble xyliquimod and S. aureus co-administered with 1 nM soluble PPX (equimolar to that conjugated to the bacteria) were included as controls. mRNA expression of IL-1β, IL-6, TNFα, IL-12p35, and IL-12p40 was assessed by qPCR. Results expressed as fold increase over unstimulated macrophages. Experiment performed in technical triplicate; error bars represent SEM.

FIG. 13 depicts experimental results demonstrating that the concentration of copper necessary for click chemistry is highly toxic to S. aureus. WT S. aureus Newman was grown in the presence and absence of 2 mM copper for 4.25 hours. Growth was monitored by optical density: absorbance measurements were taken every 15 minutes at 600 nm.

FIG. 14, comprising FIG. 14A through FIG. 14C, depicts experimental results demonstrating that CPX, a cyclooctyne-functionalized derivative of xyliquimod, activates TLR7 and augments immune responses when conjugated to S. aureus. FIG. 14A depicts the structure of CPX: a xyliquimod derivative attached via a PEGS linker to an activated cyclooctyne, which allows copper-free click ligation to azides. FIG. 14B depicts the activity of soluble CPX in primary macrophages. WT and TLR7KO murine bone marrow-derived macrophages were stimulated with various concentrations of soluble CPX for 6 hours and mRNA expression of IL-1β was assessed by qPCR. FIG. 14C depicts CPX was adsorbed or conjugated to heat-killed S. aureus at various concentrations, and the modified bacteria were used to stimulate macrophages for 6 hours (20 MOI). mRNA expression of IL-1β was assessed by qPCR. Results are expressed as fold increase over unstimulated macrophages. Experiments performed in technical triplicate; error bars represent SEM.

FIG. 15, comprising FIG. 15A through FIG. 15C, depicts experimental results demonstrating that CPX2, a more polar variant of CPX, stimulates stronger immune responses than CPX when conjugated to S. aureus. FIG. 15A depicts the structure of CPX2: a xyliquimod derivative attached to an activated cyclooctyne via an amino acid with a diacid side chain and a PEGS linker. FIG. 15B depicts the activity of soluble CPX2 in primary macrophages. Murine bone marrow-derived macrophages were stimulated with various concentrations of soluble CPX for 6 hours and mRNA expression of IL-6 was assessed by qPCR. FIG. 15C depicts CPX or CPX2 was adsorbed or conjugated to heat-killed S. aureus (at 100 nM), and modified bacteria were used to stimulate macrophages for 12 hours (20 MOI). mRNA expression of IL-1β was assessed by qPCR. Results expressed as fold increase over unstimulated macrophages. Experiments performed in technical triplicate; error bars represent SEM.

FIG. 16, comprising FIG. 16A and FIG. 16B, depicts experimental results demonstrating that CPX2 conjugation to S. aureus stimulates TLR7-dependent expression of pro-inflammatory cytokines, type I IFN, and Th1-promoting cytokines in primary macrophages. FIG. 16A depicts mRNA expression of IL-1β. WT and TLR7KO murine bone marrow-derived macrophages were stimulated with heat-killed S. aureus conjugated to CPX2 (2 MOI). Untreated S. aureus, CPX2-adsorbed S. aureus, and S. aureus co-administered with 100 nM soluble CPX2 (100-fold in excess of that conjugated to bacteria) were included as controls. mRNA expression of IL-1β was assessed by qPCR. FIG. 16B depicts mRNA expression of IFNβ and IL-12p40WT macrophages were stimulated with modified bacteria for 6 hours (10 MOI) and mRNA expression of IFNβ and IL-12p40 was assessed by qPCR. Results are expressed as fold increase over unstimulated macrophages. Experiments performed in technical triplicate; error bars represent SEM.

FIG. 17, comprising FIG. 17A and FIG. 17B, depicts experimental results demonstrating that CPX2 conjugation to S. aureus stimulates TLR7-dependent expression of pro-inflammatory cytokines and Th1-promoting cytokines in primary dendritic cells. WT and TLR7KO murine bone marrow-derived dendritic cells were stimulated with heat-killed S. aureus conjugated to CPX2 for 6 hours (2 MOI). Untreated S. aureus, CPX2-adsorbed S. aureus, and S. aureus co-administered with 100 nM soluble CPX2 (100-fold in excess of that conjugated to bacteria) were included as controls. FIG. 17A depicts mRNA expression of IL-1β and FIG. 17B depicts mRNA expression of IL-12p40. mRNA expression was assessed by qPCR. Results are expressed as fold increase over unstimulated dendritic cells. Experiment performed in technical triplicate; error bars represent SEM.

FIG. 18, comprising FIG. 18A through FIG. 18C, depicts experimental results demonstrating that CPX2 conjugation to S. aureus increases expression of maturation markers in primary dendritic cells via TLR7. WT and TLR7KO murine bone marrow-derived dendritic cells were stimulated for 24 hours with heat-killed S. aureus conjugated to CPX2 (10 MOI). Untreated heat-killed S. aureus was included as a control. FIG. 18A depicts Surface expression of CD86. FIG. 18B depicts the surface expression, representative flow cytometry trace and relative quantitation of CD80. FIG. 18B depicts the surface expression, representative flow cytometry trace and relative quantitation of MHCII. Data are expressed as an increase in mean fluorescence over unstimulated dendritic cells. Experiments performed in technical duplicate; error bars represent SEM.

FIG. 19, comprising FIG. 19A and FIG. 19B, depicts the quantification of molecules conjugated to S. aureus via click chemistry. FIG. 19A depicts the structures of clickable fluorescein derivatives: alkynyl fluorescein and cyclooctynyl fluorescein. FIG. 19B depicts the standard curve generated using the MESF FITC quantitation kit and QuickCal 2.3.

### DETAILED DESCRIPTION OF THE INVENTION

The present invention provides compositions and methods for treating or preventing a disease or disorder in a subject. The present invention is based, in part, upon the discovery that conjugation of an immunomodulatory molecule to the surface of a cell greatly improves the ability of the cell to activate immune cells. In certain embodiments, the cell is a bacterial cell.

In one embodiment, the present invention provides a composition for the treatment and prevention of a microbial infection or a disease or disorder associated with a microbial infection. In one embodiment, the present invention provides a composition for the treatment and prevention of a microbial infection or a disease or disorder associated with the infection of a pathogenic bacterium. In one embodiment, the composition comprises a cell comprising an immunomodulatory molecule bound to the cell surface. For example, in one embodiment, the invention provides a bacterial cell having an immunomodulatory molecule covalently bound to the cell surface

In one embodiment, the present invention provides a method of inducing an immune response against a pathogen. In one embodiment, the present invention provides a method of the present invention provides a method a microbial infection. In some embodiments, the method comprises administering a composition comprising a cell of the invention.

### Definitions

Unless defined otherwise, all technical and scientific terms used herein have the same meaning as commonly understood by one of ordinary skill in the art to which this invention belongs.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample.

“Linker” refers to one or more atoms comprising a chain connecting two chemical moieties.

“Cell lysate” refers to a solution produced when the cell membranes or cell walls of cells are disrupted, either by physical or chemical methods. In some embodiments, cell lysate is prepared from a bacterial cell. In some embodiments, cell lysate is prepared from a bacterial cell having an immunomodulatory molecule bound to the cell surface. In some embodiments, cell lysate is produced under a number of conditions, including repeated freezing and thawing, homogenizing, contacting with a hyper- or hypo-tonic solution or contacting with one or more non-ionic detergents.

By the term “vector” as used herein, is meant any plasmid or virus encoding an exogenous nucleic acid. The term should also be construed to include non-plasmid and non-viral compounds which facilitate transfer of nucleic acid into cells, or organisms.

As used herein, by “combination therapy” is meant that a first agent is administered in conjunction with another agent. “In conjunction with” refers to administration of one treatment modality in addition to another treatment modality. As such, “in conjunction with” refers to administration of one treatment modality before, during, or after delivery of the other treatment modality to the individual. Such combinations are considered to be part of a single treatment regimen or regime.

As used herein, the term “concurrent administration” means that the administration of the first therapy and that of a second therapy in a combination therapy overlap with each other.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

The term “immunoglobulin” or “Ig,” as used herein, is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4⁺, CD8⁺, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for inducing an immune response against a pathogen. In some embodiments, the invention relates to compositions and methods for the treatment, inhibition, prevention, or reduction of an infection. In some embodiments, the invention relates to compositions and methods for the treatment, inhibition, prevention, or reduction of a microbial infection or disease or disorder associated with a microbial infection.

In one embodiment, the invention provides a modified cell comprising an immunomodulatory molecule bound to the cell surface. Conjugation of the immunomodulatory molecule to the cell surface allows the cell to activate immune cells as the immunomodulatory molecule in combination with the naturally polyantigenic cell produce powerful T cell-responses there by improving the immunogenicity of the cell itself. Accordingly, the invention also provides immunogenic compositions comprising the modified cells of the invention.

In various embodiments, the invention is directed towards modified bacterial cells comprising an immunomodulatory molecule bound to the cell surface. For example, in some embodiments, the modified cell can be a Staphylococcus sp. cell. In some embodiments, the immunomodulatory molecule has Toll-like receptor (TLR) agonist activity. For example, in various embodiments, the immunomodulatory molecule comprises an imidazoquinoline derivative.

### Cells and Vectors

In one aspect, the present invention provides a modified cell or vector comprising an immunomodulatory molecule bound to the cell surface. The immunomodulatory molecule can be bound to the cell or vector surface by any method known in the art. For example, in one embodiment the immunomodulatory molecule is covalently linked to the cell or vector surface. In one embodiment, the immunomodulatory molecule is linked to the cell or vector surface through a functional group. In one embodiment, the immunomodulatory molecule is linked to the cell or vector surface through a click-chemistry reaction. In one embodiment, the immunomodulatory molecule is linked to the cell or vector surface through sortase-mediated incorporation.

In one embodiment, the present invention provides a modified cell comprising an immunomodulatory molecule bound to the cell surface. In one embodiment the cell is a prokaryotic cell. In one embodiment the cell is a bacterial cell. In one embodiment, the bacterial cell is gram positive. In one embodiment, the bacterial cell is gram negative. In some embodiments, the bacteria is from a genus including, but not limited to, Actinomyces spp., Bacteroides spp., Clostridium spp., Corynebacterium spp., Enterobacter spp., Enterococcus spp., Fusobacterium spp., Klebsiella spp., Legionella spp., Leptospirosis spp., Morganella spp., Peptococcus spp., Prevotella spp., Proteus spp., Providentia spp., Pseudomonas spp., Staphylococcus spp., or Streptococcus spp. For example, in some embodiments, the bacteria includes, but is not limited to, Actinomyces spp., anaerobic bacteria, anaerobic streptococci, Bacillus cereus, Bacteroides fragilis, Bacteroides thetaiotaomicron, Bacteroides vulgatus, Beta hemolytic streptococci (group A, B, C or G), Bordetella pertussis, Borrelia burgdorferi, Chlamydia trachomatis, Chlamydophila pneumoniae, Clostridium difficile, Clostridium perfringens, coagulase-negative staphylococci, Corynebacterium diptheriae, Corynebacterium ulcerans, Enterococcus faecalis, Escherichia coli, Fusobacterium nucleatum, Gardenerella vaginalis, Haemophilus influenzae, Helicobacter pylori, Hemophilus influenza, Klebsiella pneumoniae, Listeria monocytogenes, Moraxella catarrhalis, Mycobacterim tuberculosis, Mycobacterium avium, Mycobacterium tuberculosis, Mycoplasma pneumoniae, Mycoplasma spp., Neisseria gonorrhea, Neisseria gonorrhoeae, Neisseria meningitidis, Peptostreptococcus spp., Prevotella melaninogenica, Prevotella melaninogenicus, Proteus mirabilis, Proteus vulgatus, Pseudomonas aeruginosa, S. anginosus, S. bovis, S. constellatus, S. intermedius, S. mutans, S. sanguis, Salmonella enteriditis, salmonella species, Salmonella spp., Salmonella typhi, Serratia spp., Shigella flexneri, Staphylococcus aureus, Streptococcus (anginosus group), Streptococcus agalactiae, Streptococcus milleri, Streptococcus mutans, Streptococcus pneumoniae, Streptococcus pyogenes, Streptococcus salivarius, Streptococcus sanguis, Treponema pallidum, viridans streptococci, Yersinia enterocolitica, Yersinia pseudotuberculosis, and α-streptococci. In one embodiment, the cell is a Staphylococcus aureus cell.

In one embodiment, the cell is a fungal cell. For example, in some embodiments, the fungus includes, but is not limited to Candida, Aspergillus, Cryptococcus, Coccidioides, Histoplasma, Pneumocystis, Blastomyces, Ringworm, Sporothrix, Mucormycosis, Exserohilum, Cladosporium and Stachybotrys.

In one embodiment, the present invention provides a modified vector comprising an immunomodulatory molecule bound to the vector surface. In one embodiment the vector is a virus. In one embodiment, the virus is a pathogenic virus. Exemplary viruses include, but are not limited to, HIV-1, HIV-2, hepatitis virus (e.g. hepatitis A, hepatitis B, hepatitis C, hepatitis D, and hepatitis E viruses), herpes simplex viruses (e.g. HSV1 and HSV2), varicella-zoster virus, cytomegalovirus, Epstein Barr virus, and human herpes viruses (e.g. HSV6, HSV7, HSV8), respiratory syncytial viruses, filoviruses (e.g. Ebolavirus and Marburg virus), smallpox viruses, Chikungunya viruses, foot and mouth disease viruses, influenza viruses (e.g. influenza A and influenza B) and flaviviruses (e.g. yellow fever virus, west nile virus, dengue fever virus and zika virus).

In some embodiments, the cell or vector is inactivated or killed. Inactivated or killed generally refers to infectious agents (e.g., bacteria, viruses, other microorganisms or agents) that are not capable of reproducing or causing full disease (i.e., avirulent), but are still able to initiate or stimulate an immune response. Inactivated bacterial preparations may be called bacterins. The inactivated or killed agents are able to initiate or stimulate an immune response when administered to an animal, in the context of a vaccine composition, for example. In contrast to inactivated or killed vaccines, live vaccines and live-attenuated vaccines, for example, are able to replicate and generally do so once they are administered to an animal. In some embodiments, the cell is live-attenuated.

The cells or vectors may be killed or inactivated using a variety of methods. In one example, the cells or vectors may be treated with various chemicals for various periods of time to render the agents incapable of replication, but still retaining at least some ability to stimulate an immune response (i.e., immunogenicity) when administered to an animal. Many such agents are known. Exemplary inactivating agents include, but are not limited to formalin/formaldehyde, ethyleneimine derivatives, ultraviolet radiation or heat, thimerosal and/or β-propiolactone, and others. The infectious agents are generally treated with a concentration of the agent for a length of time and at a temperature to inactivate the cell, yet still preserve at least some of the ability of the agent to be immunogenic and stimulate an immune response. Inactivating agents may be removed prior to formulation into compositions for administration to animals.

In certain embodiments, the composition comprises an attenuated cell. For example, in one embodiment, the cell may be temperature-sensitive. In one embodiment, the cell may be cold-adapted. In certain aspects, these three features are particularly useful when using live cells as a vaccine antigen. In certain instances, compared with an inactivated vaccine, an attenuated vaccine can offer great advantages in activating immune responses in a host. First, as a live replicating cell in the host, the attenuated vaccine strain can be readily detected by the immune system, and thereby can activate a wide spectrum of immune responses. Second, since attenuated vaccine strain can replicate in host, they continuously present antigens to the immune system and therefore provides durable immunity and requires less often vaccination or boosters.

In one embodiment, the immunomodulatory molecule comprises an adjuvant group, a linker group and a targeting group. In one embodiment, the adjuvant group enhances elicited immune responses (humoral and/or cellular).

In one embodiment, the targeting group is capable of participating in a click-chemistry reaction. For example, in one embodiment, the targeting group comprises a terminal alkyne, a terminal azide, or a cyclooctyne group. In one embodiment, the targeting portion is capable of participating in a sortase-mediated labeling reaction. For example, in one embodiment, the targeting group comprises a sortase targeting peptide. In one embodiment, the sortase targeting peptide comprises the amino acid sequence LPETG (SEQ ID NO:1).

In one embodiment, the linker group bridges the adjuvant group and the targeting group. In one embodiment, the linker group is capable of being cleaved, thereby allowing for the adjuvant portion to be cleaved from the cell surface. For example, in one embodiment, the linker group comprises a disulfide group. Accordingly, the disulfide linker would be cleaved in the reducing environment of the phagosome after uptake of the chemically modified bacterium by an APC and the thiol-derivative of the adjuvant group would be released from the cell wall.

Exemplary linker groups may include, but are not limited to, a disulfide linker group, a thioester group, one of various acid-labile linkers, and a polyethylene glycol (PEG) group.

In one embodiment, the adjuvant group comprises an immunostimulatory moiety. Exemplary immunostimulatory moieties include, but are not limited to, imidazoquinoline, thiazoloquinolone, trehalose-6,6-dibehenate (TDB), Cyclic [G(2′,5′)pA(2′,5′)p], Cyclic [G(2′,5′)pA(3′,5′)p], Cyclic [G(3′,5′)pA(3′,5′)p], Cyclic diadenylate monophosphate (c-di-AMP), 2′3′-c-di-AM(PS)₂ (Rp,Rp), Cyclic diguanylate monophosphate (c-di-GMP), Poly(D-glucosamine), [S-(2,3-bis(palmitoyloxy)-(2RS)propyl)-(R)-cysteinyl 4-((6-amino-2(butyl amino)-8-hydroxy-9H-purin-9-yl)methyl)aniline], [S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl)methyl)aniline], Pam2C-Aca-Benzyl-Murabutide, and derivatives thereof.

In one embodiment, the adjuvant group has Toll-like receptor (TLR) agonist activity. For example, in one example, the adjuvant group activates TLR 3, TLR 7, TLR 8, TLR 7/8, TLR 9, or any combination thereof. For example, in one embodiment, the adjuvant group comprises an imidazoquinoline or derivative thereof, CpG oligodeoxynucleotides, a thiazoloquinolone or derivative thereof. Exemplary imidazoquinoline derivatives include, but are not limited to imiquimod, resiquimod, gardiquimod, and imidazoquinoline CL097. Exemplary thiazoloquinolone derivatives include, but are not limited to, CL075.

In some embodiments, the immunomodulatory molecule includes, but is not limited to:

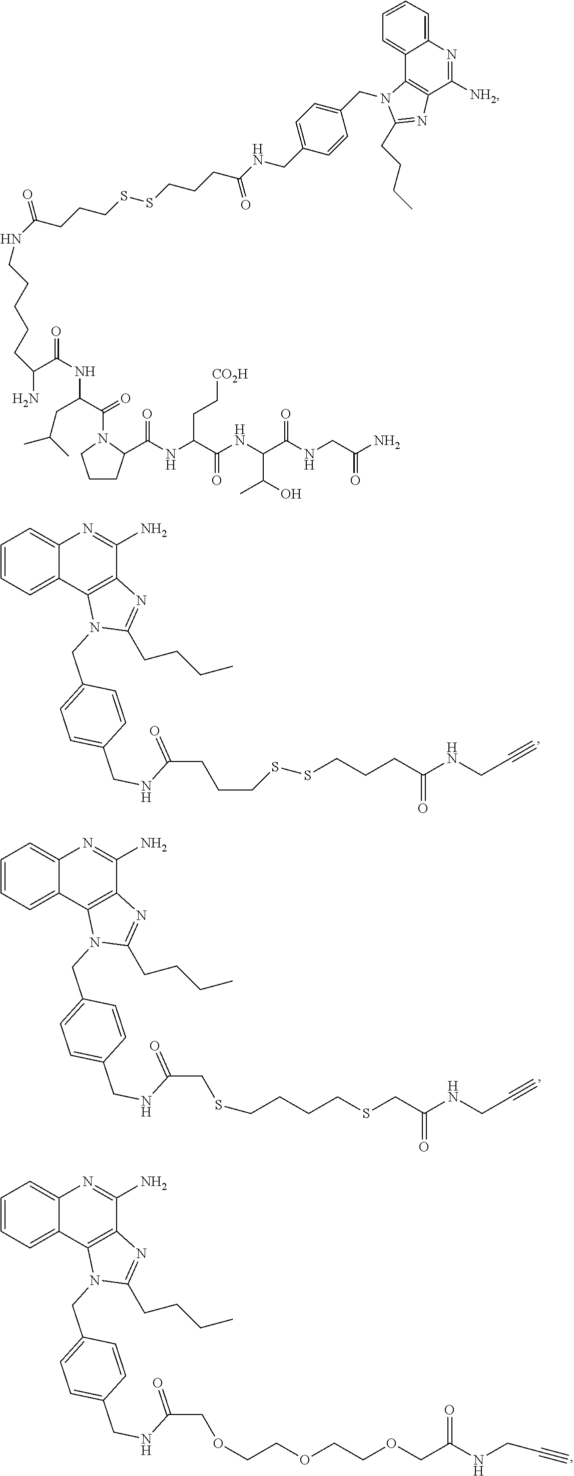

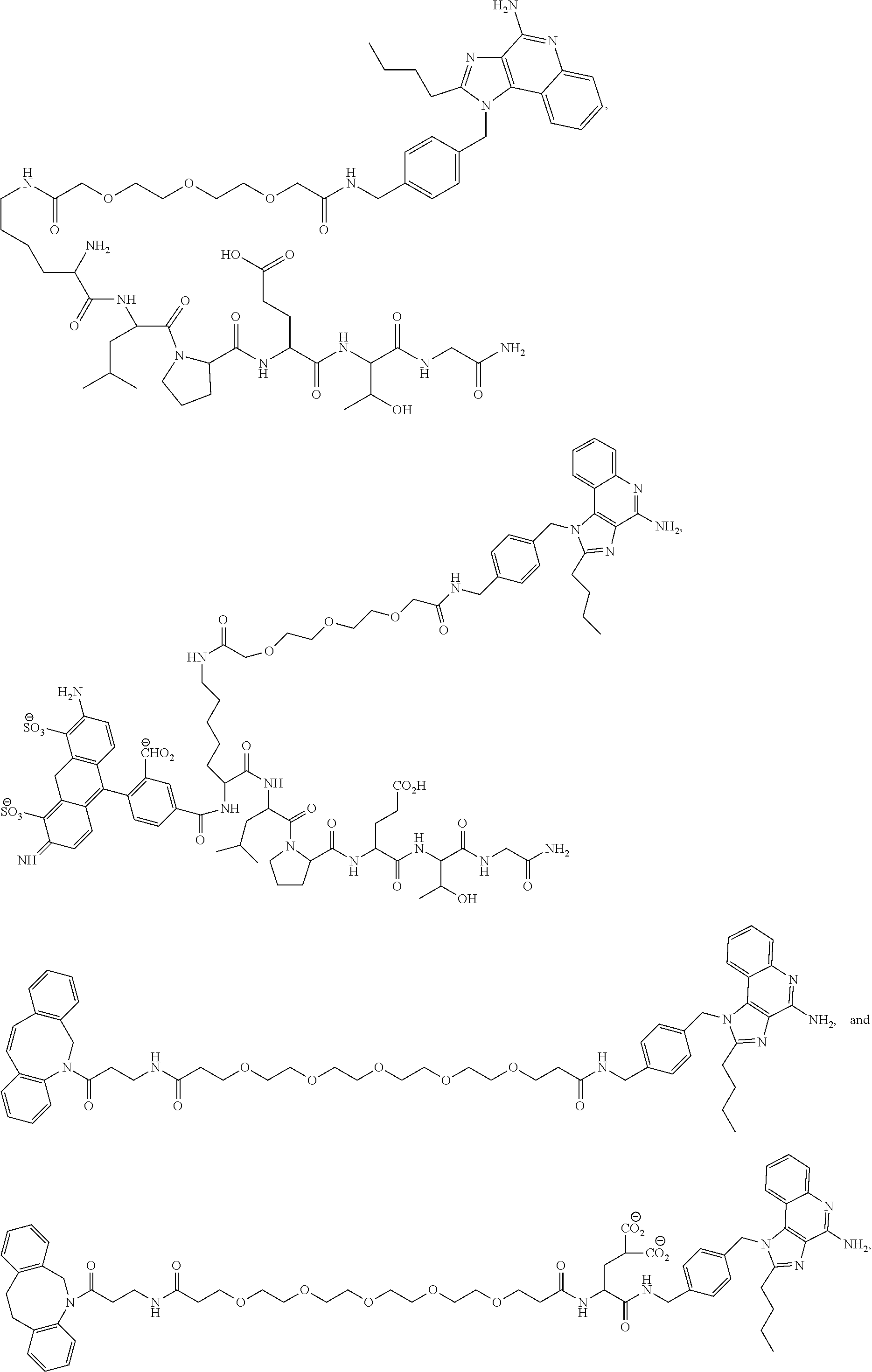

and any derivative thereof.

Cells or vectors generated by the approaches described herein can be used in the vaccine and pharmaceutical formulations described herein. Reverse genetics techniques can also be used to engineer mutations to genes important for vaccine production—i.e., the epitopes of useful vaccine strain variants can be engineered into the bacterium. Alternatively, completely foreign epitopes, including antigens derived from other pathogens can be engineered into the inactivated or attenuated strain.

The inactivated or attenuated cell or vector of the present invention can itself be used as the active ingredient in vaccine or pharmaceutical formulations. In certain embodiments, the bacterium can be used as the vector or “backbone” of recombinantly produced vaccines. To this end, the “reverse genetics” technique can be used to engineer mutations or introduce foreign epitopes into the cell, which would serve as the “parental” strain. In this way, vaccines can be designed for immunization against strain variants, or in the alternative, against completely different infectious agents or disease antigens.

For example, in one embodiment, the immunological composition of the invention comprises a cell or vector, having an immunomodulatory molecule bound to the cell surface, engineered to express one or more epitopes or antigens of a given pathogen. For example, the cell can be engineered to express neutralizing epitopes of other preselected strains. Alternatively, epitopes of other pathogens can be built into the engineered cell.

In one embodiment, the cell or vector is capable of inducing a robust immune response in the host—a feature which contributes to the generation of a strong immune response when used as a vaccine, and which has other biological consequences that make the cell useful as pharmaceutical agents for the prevention and/or treatment of an infection, disease, or disorder associated with an antigen.

### Compositions

The present invention provides immunological compositions that, when administered to a subject in need thereof, elicit an immune response directed against a pathogen. Further, when the compositions are administered to a subject, they elicit an immune response that serves to protect the inoculated subject against the pathogen. For example, in one embodiment, when the compositions are administered to a subject, they elicit an immune response directed against a pathogenic bacteria.

In one embodiment, the present invention provides compositions that are useful as immunomodulatory agents, for example, in stimulating immune responses and in preventing infection of a pathogen and diseases or disorders associated with infection of a pathogen.

In various embodiments, the composition comprises a cell of the invention, cell lysate of a cell of the invention or a vector of the invention. For example, in some embodiments, the composition comprises a cell comprising an immunomodulatory molecule bound to the cell surface. In some embodiments, the composition comprises the lysate of a cell comprising an immunomodulatory molecule bound to the cell surface. In one embodiment, the composition comprises a bacterial cell comprising an immunomodulatory molecule bound to the cell surface. In some embodiments, the composition comprises the lysate of a bacterial cell comprising an immunomodulatory molecule bound to the cell surface.

In one embodiment, the bacterial cell is a pathogenic bacterial cell. In certain embodiments, the bacterial cell is unable to replicate, but is still able to induce an anti-microbial immune response. In certain embodiments, the bacterial cell is an inactivated bacterial cell.

In certain embodiments, the composition may reduce or prevent the replication of the bacteria in a subject.

In one embodiment, the present invention provides compositions that are useful as immunomodulatory agents, for example, in stimulating immune responses and in preventing a microbial infection and microbial infection-related pathology.

### Vaccines

In the context of the present invention, the term “vaccine” (also referred to as an immunogenic composition) refers to a substance that induces an immune response upon inoculation into a subject. In certain embodiments, the vaccine induces an adaptive immune response. In some instances, the vaccine of the invention can be used to induce an immune response against an immunogen, thereby depleting the immunogen and treating or preventing a disease or disorder. For example, in one embodiment, the vaccine of the invention can be used to induce an immune response against pathogenic bacteria, thereby depleting the immunogen and treating or preventing a disease or disorder associated with the pathogenic bacteria.

In one embodiment, the composition comprises a vaccine, where the vaccine induces an immune response to one or more antigens in a cell, tissue or mammal (e.g., a human). In certain embodiments, the vaccine may comprise an antigen (e.g., a peptide or polypeptide), a nucleic acid encoding an antigen (e.g., an antigen expression vector), a cell expressing or presenting an antigen or cellular component. In particular embodiments the vaccine comprises a cell having an immunomodulatory molecule bound to the cell surface as described herein, or an immunologically functional equivalent thereof.

In some embodiments, the composition comprises an additional immunomodulatory agent or nucleic acids encoding such an agent. Immunomodulatory agents include but are not limited to an additional antigen, an immunomodulator, an antigen presenting cell or an adjuvant.

Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

In one embodiment, the vaccine of the invention includes, but is not limited to, a cell or vector having an immunomodulatory molecule bound to the cell or vector surface. The immunomodulatory molecule bound to the cell surface improves the vaccine's ability to activate innate immune cells, and induce a robust type I interferon response. For example, in some embodiments the vaccine of the invention includes a bacterium having an immunomodulatory molecule bound to the cell surface. Exemplary immunomodulatory molecules include, but are not limited to derivatives of imidazoquinoline, and derivatives of thiazoloquinolone.

A vaccine of the present invention may vary in its composition of cellular components. In a non-limiting example, cell might also be formulated with an additional adjuvant.

When a certain modified bacterial cell or combination of modified bacterial cells of the invention induce an immune response upon inoculation into an animal, certain modified cell or combination of modified cells are decided to have anti-bacterial immunity inducing effect. The induction of the anti-bacterial immunity by a modified bacterial cell or combination of modified bacterial cells can be detected by observing in vivo or in vitro the response of the immune system in the host against the modified cell.

For example, a method for detecting the induction of cytotoxic T lymphocytes is well known. A foreign substance that enters the living body is presented to T cells and B cells by the action of APCs. T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by a certain modified bacterial cell or combination of modified bacterial cells of the invention can be evaluated by presenting the peptide to a T cell by APC, and detecting the induction of CTL. Furthermore, APCs have the effect of activating CD4+ T cells, CD8+ T cells, macrophages, eosinophils and NK cells.

A method for evaluating the inducing action of CTL using dendritic cells (DCs) as APC is well known in the art. DC is a representative APC having the strongest CTL inducing action among APCs. In this method, the modified bacterial cell or combination of modified bacterial cells are initially contacted with DC and then this DC is contacted with T cells. Detection of T cells having cytotoxic effects against the cells of interest after the contact with DC shows that the modified bacterial cell or combination of modified bacterial cells have an activity of inducing the cytotoxic T cells. Furthermore, the induced immune response can be also examined by measuring IFN-gamma produced and released by CTL in the presence of antigen-presenting cells that carry immobilized antigens of the modified cell by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The modified bacterial cell or combination of modified bacterial cells confirmed to possess CTL inducing activity by these methods are cells having DC activation effect and subsequent CTL inducing activity. Therefore, a cell or combination of cells that induce CTL against a pathogenic bacteria are useful as vaccines to deplete the pathogenic bacteria thereby useful for treating or preventing an disease or disorder associated with the pathogenic bacteria. Furthermore, CTL that have acquired cytotoxicity due to presentation of the antigens of the modified bacterial cell by APC can be also used as vaccines to deplete a pathogenic bacteria.

The induction of anti-microbial immunity by a modified bacterial cell or combination of modified bacterial cells can be further confirmed by observing the induction of antibody production against the bacteria. For example, when antibodies against a cell or combination of cells are induced in a laboratory animal immunized with the cell or combination of cells, and when the bacteria is depleted in the animal, the cell or combination of cells are determined to induce anti-microbial immunity.

In certain embodiments, the composition comprises an inactivated or killed bacterium. Inactivated or killed generally refers to infectious agents (e.g., bacteria, viruses, other microorganisms or agents) that are not capable of reproducing or causing disease (i.e., avirulent). Inactivated bacterial preparations may be called bacterins. The inactivated or killed agents are able to stimulate an immune response when administered to an animal, in the context of a vaccine composition, for example. In contrast to inactivated vaccines, live vaccines and live attenuated vaccines, for example, are able to replicate and generally do so once they are administered to an animal. In some embodiments, the composition comprises a live attenuated vaccine.

Another type of vaccine, called subunit vaccines, also does not replicate. Subunit vaccines generally contain substantially less than all of a bacterium or virus and, in this way, often may be distinguished from inactivated/killed vaccines. For example, subunit vaccines may contain single or a few recombinant protein antigens from a bacterium or virus. Subunit vaccines may also contain individual structures, like a capsid or capsomere from a virus, for example. Inactivated or killed vaccines generally include more of a bacterium or virus, for example, than does a subunit vaccine. For example, an inactivated vaccine may contain all or substantially all of a virus or bacterium. In one example, entire cultures of bacteria or viruses may be inactivated or killed. In another example, less than all, but still substantial parts of bacteria or viruses may be used in an inactivated/killed vaccine. For example, bacteria may be extracted with a chemical to obtain the cell wall, cell membrane or cell wall plus cell membrane portions that may be used as or in an inactivated/killed vaccine or immune stimulatory composition.

Generally, agents for inclusion in an inactivated or killed vaccine may be grown, purified or semi-purified, inactivated, and then formulated into a vaccine composition. Bacteria may be grown on cell free, serum-free, protein-free, synthetic medium and the like, using commonly known methods for growth of pure bacterial cultures. Often, bacteria are grown in liquid cultures. The bacteria may be purified, semi-purified, and/or concentrated. For example, bacteria grown in liquid culture may be subject to relatively low-speed centrifugation, the culture medium decanted, and the bacterial pellet re-suspended in buffer.

The bacteria may be killed or inactivated using a variety of methods. In one example, the bacteria may be treated with various chemicals for various periods of time to render the agents incapable of replication, but still retaining at least some ability to stimulate an immune response (i.e., immunogenicity) when administered to an animal. Many such agents are known. Example inactivating agents include, but are not limited to formalin/formaldehyde, ethyleneimine derivatives, ultraviolet radiation or heat, thimerosal and/or β-propiolactone, and others. The infectious agents are generally treated with a concentration of the agent for a length of time and at a temperature to inactivate the bacteria, yet still preserve at least some of the ability of the agent to be immunogenic and stimulate an immune response. Inactivating agents may be removed prior to formulation into compositions for administration to animals.

In certain embodiments, the composition comprises an attenuated Staphylococcus sp. bacterium. For example, in one embodiment, the bacterium may be temperature-sensitive. In one embodiment, the bacterium may be cold-adapted. In certain aspects, these three features are particularly useful when using live bacteria as a vaccine antigen. In certain instances, compared with an inactivated vaccine, an attenuated vaccine can offer great advantages in activating immune responses in a host. First, as a live replicating bacteria in the host, the attenuated vaccine strain can be readily detected by the immune system, and thereby can activate a wide spectrum of immune responses. Second, since attenuated vaccine strain can replicate in host, they continuously present antigens to the immune system and therefore provides durable immunity and requires less often vaccination or boosters.

An anti-microbial immune response can be induced by administering a vaccine of this invention, and the induction of an anti-microbial immune response enables treatment and prevention of a disease or disorder associated with the pathogenic bacteria.

The cell or combination of cells of the invention having immunological activity, may be combined with an additional adjuvant. An adjuvant refers to a compound that enhances the immune response against the cell or combination of cells when administered together (or successively) with the cell having immunological activity. Examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such.

### Pharmaceutical Compositions

The present invention envisions treating or preventing a bacterial infection or bacterial infection-related pathology in a mammal by the administration of a therapeutic composition of the invention to a mammal in need thereof. Administration of the composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

The present invention encompasses pharmaceutical compositions comprising a modified bacterial cell to be used as antibiotic agents or as agents against bacterial infection-related diseases and disorders. The pharmaceutical compositions have utility as an antibiotic prophylactic and may be administered to a subject at risk of getting infected or is expected to be exposed to a bacterium.

The mutant modified cells and vectors of the invention may be engineered using the methods described herein to have an immunomodulatory molecule bound to the cell surface which improve the immunogenicity of the cell or vector. In one embodiment, the modified cell of the invention comprises an imidazoquinoline derivative attached to the cell surface.

Methods of introduction include but are not limited to intradermal, intramuscular, intraperitoneal, intravenous, subcutaneous, intranasal, epidural, and oral routes. The pharmaceutical compositions of the present invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in an exemplary embodiment it may be desirable to introduce the pharmaceutical compositions of the invention into the lungs by any suitable route. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In one embodiment, the pharmaceutical composition can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In one embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger & Peppas, 1983, J. Macromol. Sci. Rev. Macromol. Chem. 23:61; see also Levy et al., 1985, Science 228:190; During et al., 1989, Ann. Neurol. 25:351 (1989); Howard et al., 1989, J. Neurosurg. 71:105). In one embodiment, a controlled release system can be placed in proximity of the composition's target, i.e., the lung, thus requiring only a fraction of the systemic dose (see, e.g., Goodson, 1984, in Medical Applications of Controlled Release, supra, vol. 2, pp. 115-138). Other controlled release systems are discussed in the review by Langer (1990, Science 249:1527-1533).

The pharmaceutical compositions of the present invention comprise a therapeutically effective amount of the attenuated virus, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water and the like. These compositions can take the form of solutions, suspensions, emulsion, tablets, pills, capsules, powders, sustained-release formulations and the like. These compositions can be formulated as a suppository. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

The amount of the pharmaceutical composition of the invention which will be effective in the treatment or prevention of a particular disease or disorder will depend on the nature of the disease or disorder, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated.

### Methods of Treatment

The present invention provides a method for treating or preventing an infection or an infection-related disease or disorder. In one embodiment, the infection is a microbial infection, a viral infection or a fungal infection. In one embodiment, the infection is an infection of a pathogenic bacteria. Accordingly, in some embodiments, present invention provides a method for treating or preventing a pathogenic bacterial infection or a pathogenic bacterial infection-related disease or disorder. In one embodiment, the method comprises administering an immunological composition comprising a modified cell comprising an immunomodulatory molecule bound to the cell surface. In one embodiment, the method comprises administering an immunological composition comprising a modified bacterial cell comprising an immunomodulatory molecule bound to the cell surface. In one embodiment, the modified bacterial cell is a modified S. aureus cell. In one embodiment, the method comprises administering an immunological composition comprising a modified vector comprising an immunomodulatory molecule bound to the vector surface.

In certain embodiments, the modified cell or vector induces an enhanced immune response as compared to an unmodified cell. For example, in certain embodiments, the induced immune response of the modified cell is 2-fold more, 3-fold more, 5-fold more, 10-fold more, 15-fold more, 20-fold more, 50-fold more, 100-fold more, 500-fold more, or 1000-fold more, than an unmodified cell. The immune response induced by the modified cell can be measured using standard assays. For example, in certain embodiments, the immune response induced by the modified cell is measured by detecting the amount of antibodies specific to the cell antigens produced in the subject following administration of modified cell.

The therapeutic compositions of the invention may be administered prophylactically or therapeutically to subjects suffering from, or at risk of, or susceptible to, developing a disease or disorder associated with an infection. In some embodiments, the disease, disease or disorder is associated with a pathogenic bacterial infection. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In certain embodiments, the subject is a mammal. For example, the subject may include, but is not limited to, a human, primate, cow, horse, sheep, pig, dog, cat, or rodent. In certain embodiments, the relative amount of active ingredient in a single dose, or the frequency of doses, will vary depending on the age, sex, or weight of subject.

In one embodiment, a patient treated or prevented in accordance with the methods provided herein is a naïve subject, i.e., a subject that does not have a disease caused by a microbial infection or has not been and is not currently infected with a microbial infection. In one embodiment, a patient treated or prevented in accordance with the methods provided herein is a naïve subject that is at risk of acquiring a microbial infection. In one embodiment, a patient treated or prevented in accordance with the methods provided herein is a patient suffering from or expected to suffer from disease associated with a microbial infection. In one embodiment, a patient treated or prevented in accordance with the methods provided herein is a patient diagnosed with a microbial infection or a disease associated therewith. In some embodiments, a patient treated or prevented in accordance with the methods provided herein is a patient infected with a microbial infection that does not manifest any symptoms of a diseases or disorders associated with a microbial infection.

In one embodiment, a patient treated or prevented in accordance with the methods provided herein is a patient experiencing one or more symptoms of a disease or disorder associated with a microbial infection. Symptoms of diseases or disorders associated with a microbial infection include, but are not limited to, body aches, fever, nausea, cough, diarrhea, vomiting, nausea, boils, folliculitis, impetigo, cellulitis, fatigue, yellow or bloody mucus, and chest pain.

In one embodiment, a patient treated or prevented in accordance with the methods provided herein is a patient infected with an bacterium including, but not limited to, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, or Yersinia bacterium. In one embodiment, a patient treated or prevented in accordance with the methods provided herein is a patient infected with a particular species of Staphylococcus. In one embodiment, a patient treated or prevented in accordance with the methods provided herein is a patient infected with Staphylococcus aureus. In accordance with such embodiments, the patients that are infected with the pathogenic bacteria may manifest symptoms of a disease or disorder associated with a pathogenic bacterial infection.

Exemplary diseases or disorders associated with a pathogenic bacterial infection treated or prevented by way of the method include, but is not limited to, meningitis, sepsis, urinary tract infection, abscess, diarrheal illness, empyema, bacterial peritonitis, septic arthritis, pharyngitis, Brazilian purpuric fever, Yersinia pestis, bacterial vaginosis, bacterial pneumonia, pasturella multocida, aeromonas hydrophila, epiglottitis, swine brucellosis, chronic bacterial prostatitis, pyomyositis, soduku and acute prostatitis.

In one embodiment, a patient treated or prevented in accordance with the methods provided herein is a patient infected with a Staphylococcus aureus infection. In one embodiment, a patient treated or prevented in accordance with the methods provided herein is a patient infected with a Methicillin-resistant Staphylococcus aureus (MRSA) infection.

Staphylococcus aureus is a ubiquitous pathogen, and sometimes is an etiological agent of a variety of conditions, ranging in severity from mild to fatal. S. aureus produces a large number of extracellular and cell-associated proteins, many of which are involved in pathogenesis, such as alpha-toxin, beta-toxin, gamma-toxin, delta-toxin, leukocidin, toxic shock syndrome toxin (TSST), enterotoxins, coagulase, protein A, fibrinogen, fibronectin binding protein and the like.

S. aureus infections are relatively difficult to treat, and invasive diseases and relapse may occur following antibiotic treatment. Additionally, MRSA strains have become more prevalent, in hospital settings and non-hospital settings, further complicating treatment of S. aureus infections. In many instances, MRSA strains are also resistant to one or more other antibiotics including aminoglycosides, tetracycline, chloramphenicol, macrolides and lincosamides.

Exemplary diseases and disorders associated with a Staphylococcus aureus infection include, but are not limited to, meningitis, folliculitis, impetigo, abscesses, cellulitis, necrotizing fasciitis, breast infections, pneumonia, blood stream infections, endocarditis, osteomyelitis, burns, eyelid infections, food poisoning, joint infections, skin infections, surgical wound infection, scalded skin syndrome and toxic shock syndrome.

In one embodiment, a patient treated or prevented in accordance with the methods provided herein is a patient infected with a culturable but difficult-to-eradicate airway pathogen. For example, in one embodiment, the patient has cystic fibrosis. Thus, in one embodiment, the compositions and methods described herein provide for personalized therapeutic vaccines.

In one embodiment, the invention provides a method of synthesis of personalized therapeutic vaccines. For example, in one embodiment, the personalized therapeutic vaccine treats a culturable but difficult-to-eradicate airway pathogen. In one embodiment, the method comprises culturing a pathogen isolated from a patient, and linking a small molecule to the surface of the pathogen. In one embodiment, the pathogen is a bacterial cell. In one embodiment, the method further comprises inactivating the pathogen. In one embodiment, the therapeutic vaccine targets the pathogen isolated from the patient.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the invention may be administered to a subject either alone, or in conjunction with another agent, including for example, one or more antibiotics, which reduces the amount or activity of a bacteria in the subject. In certain embodiments, the method comprises administering the composition of the invention in combination with a treatment for a bacterial infection.

The type and dosage of the administered antibiotic will vary widely, depending upon the nature of the autoimmune disease or disorder, the subject's medical history, the frequency of administration, the manner of administration, and the like. The initial dose may be larger, followed by smaller maintenance doses. The dose may be administered as infrequently as weekly or biweekly, or fractionated into smaller doses and administered daily, semi-weekly, etc., to maintain an effective dosage level. Exemplary antibiotics useful for use in combination with a composition of the invention include, but are not limited to, lipopeptide, fluoroquinolone, ketolide, cephalosporin, amikacin, gentamicin, kanamycin, neomycin, netilmicin, paromomycin, streptomycin, tobramycin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime cefluprenam, cefoselis, cefozopran, cefpirome, cefquinome, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, cefuracetime, imipenem, primaxin, doripenem, meropenem, ertapenem, flumequine, nalidixic acid, oxolinic acid, piromidic acid pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, ticarcillin, sulfamethizole, sulfamethoxazole, sulfisoxazole, trimethoprim-sulfamethoxazole, demeclocycline, doxycycline, minocycline, oxytetracycline, tetracycline, linezolid, clindamycin, metronidazole, vancomycin, vancocin, mycobutin, rifampin, nitrofurantoin, and chloramphenicol.

The compositions useful for practicing the invention may be administered to deliver a dose of from 1 ng/kg/day and 100 mg/kg/day. Typically, dosages which may be administered in a method of the invention to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In certain embodiments, the dosage of the compound will vary from about 0.1 μg to about 10 mg per kilogram of body weight of the mammal. In certain embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present invention may be performed by single administration or boosted by multiple administrations.

In a specific embodiment, a patient administered a modified cell in accordance with the methods provided herein is a human. In certain embodiments, a patient administered a modified cell in accordance with the methods provided herein is a human infant. In some embodiments, a patient administered a modified cell in accordance with the methods provided herein is a human toddler. In certain embodiments, a patient administered a modified cell in accordance with the methods provided herein is a human child. In other embodiments, a patient administered a modified cell in accordance with the methods provided herein is a human adult. In some embodiments, a patient administered a modified cell in accordance with the methods provided herein is an elderly human.

In some embodiments, a patient treated or prevented in accordance with the methods provided herein is a subject affected by any condition that increases susceptibility to a bacterial infection or complications or disease resulting from a bacterial infection. In other embodiments, a patient treated or prevented in accordance with the methods provided herein is a subject in which a bacterial infection has the potential to increase complications of another condition that the individual is affected by, or for which they are at risk.

The composition may be combined with an adjuvant. An adjuvant refers to a compound that enhances the immune response when administered together (or successively) with the immunological composition. Examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

Administration

In one embodiment, the methods of the present invention comprise administering an immunological composition of the invention directly to a subject in need thereof. Administration of the composition can comprise, for example, intranasal, intramuscular, intravenous, peritoneal, subcutaneous, intradermal, as well as topical administration.

Furthermore, the actual dose and schedule can vary depending on whether the compositions are administered in combination with other pharmaceutical compositions, or depending on inter-individual differences in pharmacokinetics, drug disposition, and metabolism. One skilled in the art can easily make any necessary adjustments in accordance with the exigencies of the particular situation.

### Methods of Linking a Molecule to a Cell Surface

In one aspect, the present invention provides a method for linking a small molecule to the surface of a cell. In one embodiment, the method comprises attaching an immunomodulatory molecule to the surface of a cell through a click chemistry reaction. The click chemistry reaction allows formation of a very stable covalent bond between the cell and the immunomodulatory molecule.

The click chemistry approach was originally conceived as a method to rapidly generate complex substances by joining small subunits together in a modular fashion. (See, e.g., Kolb et al., 2004, Angew Chem Int Ed 40:3004-31; Evans, 2007, Aust J Chem 60:384-95.) Various forms of click chemistry reaction are known in the art, such as the Huisgen 1,3-dipolar cycloaddition copper catalyzed reaction (Tornoe et al., 2002, J Organic Chem 67:3057-64), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions.

For two molecules to be conjugated via click chemistry, the click chemistry moieties of the molecules must be reactive with each other. For example, in that the reactive moiety of one of the click chemistry molecule can react with the reactive moiety of the second click chemistry molecule to form a covalent bond. Such reactive pairs of click chemistry moieties are well known to those of skill in the art and include, but are not limited to the moieties listed in Table 1.

TABLE 1

| First click | Second click | |
|---|---|---|
| chemistry | chemistry | |
| moiety | moiety | Mechanism |
| azide | alkyne | Cu-catalyzed [3 + 2] azide-alkyne |
| | | cycloaddition (CuAAC) |
| azide | cyclooctyne | strain-promoted [3 + 2] azide-alkyne |
| | | cycloaddition (SPAAC) |
| azide | activated alkyne | [3 + 2] Huisgen cycloaddition |
| azide | electron- | [3 + 2] cycloaddition |
| | deficient alkyne | |
| azide | aryne | [3 + 2] cycloaddition |
| tetrazine | alkene | Diels-Alder retro-[4 + 2] cycloaddition |
| tetrazole | alkene | 1,3-dipolar cycloaddition (photoclick) |
| dithioester | diene | hetero-Diels-Alder cycloaddition |
| anthracene | maleimide | [4 + 2] Diels-Alder reaction |
| thiol | alkene | radical addition (thio-click) |
| thiol | enone | Michael addition |
| thiol | maleimide | Michael addition |
| thiol | para-fluoro | nucleophilic substitution |
| amine | para-fluoro | nucleophilic substitution |

The azide alkyne Huisgen cycloaddition reaction uses a copper catalyst in the presence of a reducing agent to catalyze the reaction of a terminal alkyne group attached to a first molecule. In the presence of a second molecule comprising an azide moiety, the azide reacts with the activated alkyne to form a 1,4-disubstituted 1,2,3-triazole. The copper catalyzed reaction occurs at room temperature and is sufficiently specific that purification of the reaction product is often not required. (Rostovstev et al., 2002, Angew Chem Int Ed 41:2596; Tornoe et al., 2002, J Org Chem 67:3057.) The azide and alkyne functional groups are largely inert towards biomolecules in aqueous medium, allowing the reaction to occur in complex solutions. The triazole formed is chemically stable and is not subject to enzymatic cleavage, making the click chemistry product highly stable in biological systems.

A copper-free click reaction has been proposed for covalent modification of biomolecules in living systems. (See, e.g., Agard et al., 2004, J Am Chem Soc 126:15046-47.) The copper-free reaction uses ring strain in place of the copper catalyst to promote a [3+2] azide-alkyne cycloaddition reaction (Id.) For example, cyclooctyne is a 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions, eliminating the need for the use of copper, which may be toxic to some to living cells.

In one embodiment, the method comprises attaching a first moiety capable of participating in a click chemistry reaction to a free amine on the surface of the cell; incubating the cell with a mixture comprising an immunomodulatory molecule comprising a second moiety capable of participating in a click chemistry reaction.

In one embodiment, the step attaching a first moiety capable of participating in a click chemistry reaction to a free amine on the surface of the cell attaches an azide to a free amine on the surface of the cell. In one embodiment, the step attaching a first moiety capable of participating in a click chemistry reaction to a free amine on the surface of the cell attaches a terminal alkyne to a free amine on the surface of the cell. In one embodiment attaching a first moiety capable of participating in a click chemistry reaction to a free amine on the surface of the cell comprises treating the cell with azido-acetic acid N-hydroxysuccinimide ester (AAA-NHS). In one embodiment, azides may be attached to free thiols via maleimides.

In one embodiment, the second moiety capable of participating in a click chemistry reaction is a terminal alkyne or a cyclooctyne. When the second moiety capable of participating in a click chemistry reaction is a terminal alkyne, the reaction solution may further comprise copper. For example, in some embodiments, the solution comprises an immunomodulatory molecule comprising terminal alkyne and CuSO4.

In some embodiments, the cell is a cell that comprises a free amine. Free amines may be found, for example, within PG (at the N termini of free bridge peptides), surface proteins (on lysine residues), lipoteichoic and wall teichoic acids (on D-alanine residues). In one embodiment the cell is a bacterial cell. The cell may be living, attenuated or inactivated. In one embodiment, the cell is a heat-inactivated cell.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples are not to be construed as limiting in any way the remainder of the disclosure.

### Example 1: Chemical Conjugation of Imidazoquinoline Adjuvants to Staphylococcus aureus: A Novel Approach in Whole Cell Vaccine Design

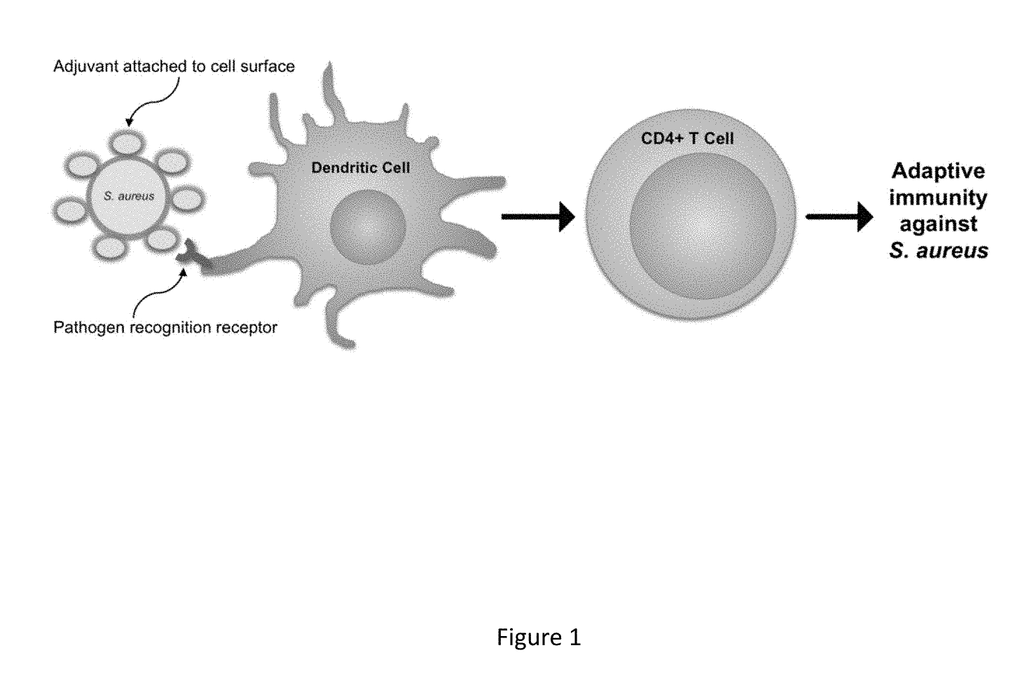

The data presented herein, demonstrates a new approach to anti-staphylococcal vaccine design, in which chemical surface displays are used to mount powerful T cell-promoting adjuvants on the surface of the naturally polyantigenic S. aureus cell (FIG. 1). These studies focus on imidazoquinolines (IMQ)—a class of adjuvants that bind to the phagosomal PRR, TLR7. IMQ possesses several immunostimulatory properties and it is hypothesized herein that IMQ would improve the immunogenicity of S. aureus.

The data presented herein establishes a chemical method for conjugating an IMQ adjuvant to the surface of heat-killed bacteria. S. aureus was selected as a model pathogen. IMQs (e.g. imiquimod) stimulate Th1 immunity through Toll-like receptor (TLR)-7 activation of antigen-presenting cells (APCs). To attach IMQ to S. aureus, azides were installed onto the bacteria using a bifunctional electrophile that reacts with primary amines within surface polymers such as proteins and glycans. Alkyne-functionalized IMQ were covalently ligated to these azides via click chemistry. To assess vaccine immunogenicity, murine APCs were exposed to IMQ-modified S. aureus and APC activation was measured via surface expression of MHCII (signal 1) and co-stimulatory markers (signal 2; CD80, CD86). Transcriptional up-regulation of T cell-polarizing (signal 3; IL-12) and pro-inflammatory cytokines (IL-1, IL-6, TNFα, IFNβ) were also measured. These data establish that IMQ-modification of S. aureus results in TLR7-dependant immunostimulation of murine APCs which allows for production of novel vaccines for both treatment of chronic infections and prevention of bacterial pneumonia.

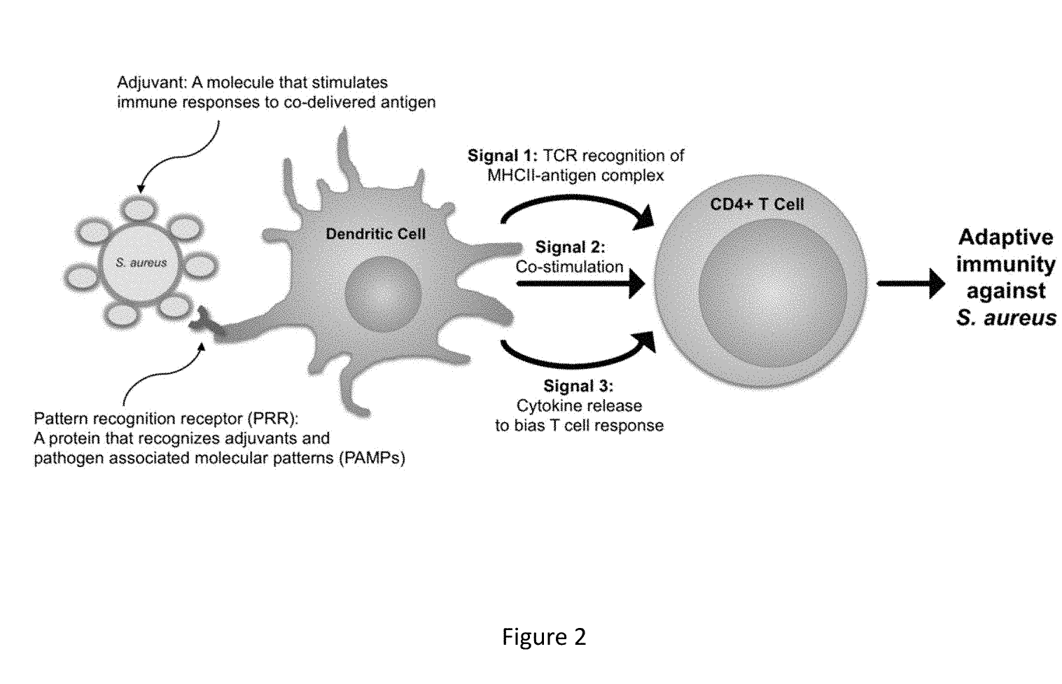

First, IMQ is known to activate pro-inflammatory NFκB and MAPK signaling in APCs through TLR7 leading to up-regulation of (i) MHCII, (ii) co-stimulatory molecules (e.g. CD80, CD86), (iii) and T cell polarizing cytokines (e.g. IL-12)—all of which promote T cell priming (FIG. 2). (Larange et al., 2009, J Leukocy Biol 85(4):673-83) Furthermore, through the well-documented synergy between IMQ and PAMPs present in the staphylococcal cell wall, including TLR2 and NOD2 ligands (Larange et al., 2009, J Leukocy Biol 85(4):673-83; Kastenmuller et al., 2011, J Clin Invest 121(5):1782-96; Ma et al., 2010, Cell Mol Immunol 7(5):381-8; Hackstein et al., 2011, Cell Immunol 271(2):401-12; Ghosh et al., 2007, Int Immunopharmacol 7(8):1111-21; Makela et al., 2009, J Leuk Biol 85(4):664-72; Schwarz et al., 2012, Immunobiology 218(4):533-42), it is hypothesized herein that conjugation of IMQ to S. aureus would potentiate CD4+ T cell responses.

Second, IMQs are known to activate an additional signaling pathway involving interferon response factors (IRFs), leading to production of type I interferons (IFNα and IFNβ). (Desmet and Ishii, 2012, Nat Rev Immunol 12(7):479-91) These cytokines seem to be generated during infection specifically with live bacteria, (Desmet and Ishii, 2012, Nat Rev Immunol 12(7):479-91; Sander et al., 2011, Nature 474(7351):385-9) which have long been shown to trigger stronger adaptive immune responses than their killed counterparts (Blander et al., 2012, Nature Rev Immunol 12(3):215-25). While little evidence exists directly linking type I IFN to the heightened protection elicited by live bacterial vaccines, they are known to promote activation of APCs and Th1 cell polarization through several well-described mechanisms. It is further hypothesized herein that, attachment of IMQ may imbue killed S. aureus with a “lifelike” immunogenicity that augments T cell immunity. In this context, it is interesting to note that S. aureus has developed immuno-evasive mechanisms to suppress IFN-I release from immune cells (Kaplan et al., 2012, J Immunol 189(9):4537-45); reversing this suppression through chemical means may therefore restore immunogenicity and promote effective adaptive immune responses. Indeed, addition of the “vita-PAMP” adjuvant, c-di-GMP (which elicits strong IFN-I responses), was shown to improve the efficacy of a vaccine against systemic S. aureus infection (Hu et al., 2009, Vaccine 27(35):4867-73).

The data presented herein demonstrates that IMQ conjugation to the surface of S. aureus greatly improves its ability to activate innate immune cells, as assessed by up-regulation of pro-inflammatory and T cell-promoting cytokines, co-stimulatory markers, and MHCII. Excitingly, a robust type I interferon response to IMQ-modified S. aureus was observed, suggesting that adjuvant conjugation did confer a measure of “life-like” immunogenicity.

Design of the Prototype Conjugatable Adjuvant, Sortase-Targeted-Disulfide-Xyliquimod

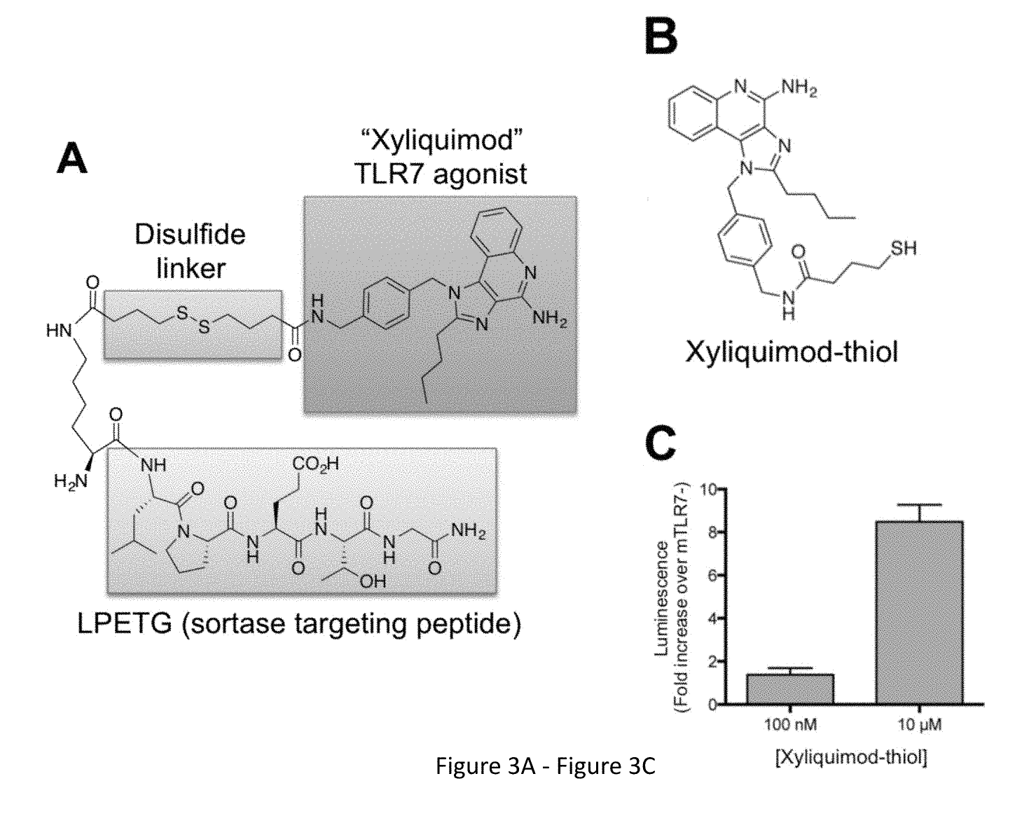

The initial approach to attaching imidazoquinoline adjuvants (IMQ) to the staphylococcal cell surface leveraged the sortase-based metabolic labeling system (FIG. 3) (Nelson et al., 2010, ACS Chem Biol 5(12):1147-55). In order to identify an IMQ derivative for attachment to LPETG (SEQ ID NO:1), the ligand, 1-(4-(aminomethyl)benzyl)-2-butyl-1Himidazo[4,5-c]quinolin-4-amine, an analog of the FDA-approved drug imiquimod (Stanley, 2002, Clin Exp Dermatol 27(7):571-7), was utilized for its potency and amenability to derivatization. Indeed, it had already been shown that the compound maintains activity at TLR7 even when conjugated to fluorophores and oligopeptides (Shukla et al., 2010, Bioorg Med Chem Lett 20(22):6384-6; Shukla et al., 2011, Bioorg Med Chem Lett 21(11):3232-6). Therefore, this molecule was selected, termed xyliquimod herein, as the TLR7-active moiety in the design of conjugatable adjuvants (FIG. 3A).

Although xyliquimod maintains activity when conjugated to peptides, and may therefore be able to ligate TLR7 even if covalently bound to peptidoglycan, it is hypothesized herein that a superior conjugation approach would be to attach the compound to the cell wall through a disulfide linker, which would be cleaved in the reducing environment of the phagosome after uptake of the chemically modified bacterium by an APC (FIG. 3A). Thus, a highly potent thiol derivative of xyliquimod (xyliquimod-thiol) would be released from the cell wall and simply diffuse to the phagosomal membrane to activate TLR7 (FIG. 3B).

To test the immuno-stimulatory activity of IMQ adjuvants such as xyliquimod-thiol, the commonly-used NFκB-luciferase reporter cell line assay was utilized. NFκB is a transcription factor that regulates immunological responses in a wide range of cell types, both immune and non-immune in lineage. In APCs, NFκB is activated in response to a number of stimuli including ligation of PRRs by adjuvants and PAMPs, and initiates an extensive genetic program that promotes adaptive immune responses. Reporter cell lines in which luciferase expression is placed under the control of NFκB is therefore an excellent means of monitoring PRR ligation and APC immune responses to experimental stimuli. In this assay, xyliquimod-thiol did indeed show robust activity at TLR7, as hypothesized above (FIG. 3C).

Determining the Optimal Linker for Xyliquimod Conjugates

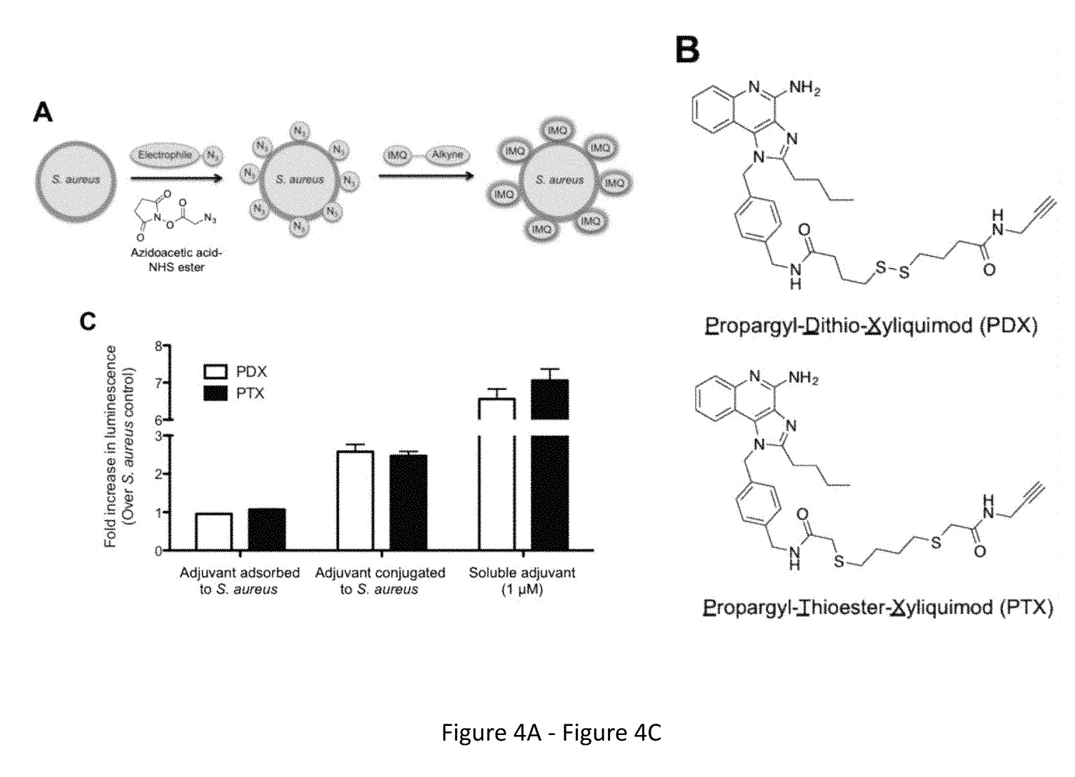

Before proceeding with the lengthy and challenging synthesis of Sortase-targeted-Disulfide-Xyliquimod (SDX, FIG. 3A), it was determined whether conjugating xyliquimod to the cell surface via a disulfide linker is in fact superior to a non-cleavable linker strategy. To test this hypothesis a simplified, “two-step” approach was used to installing compounds on the cell surface. In this approach, cells are first treated with azidoacetic acid N-hydroxysuccinimide ester (AAA-NHS) to attach azide moieties to free alkyl amines present throughout the bacterial envelope (FIG. 4A). Such amines are found within PG (at the N termini of free bridge peptides), surface proteins (on lysine residues), lipoteichoic and wall teichoic acids (on D-alanine residues), and likely other sites as well. In the second step, xyliquimod derivatives equipped with terminal alkynes are chemo-selectively ligated to the azides on the surface of S. aureus by copper-catalyzed “click” chemistry (FIG. 4A).

Using this system, it could be easily tested whether a disulfide-linked xyliquimod derivative (PDX, FIG. 4B) would have a more potent immuno-stimulatory effect than an isomeric non-cleavable xyliquimod derivative (PTX, FIG. 4B) when attached to S. aureus. Surprisingly, the cleavable linker strategy did not prove superior, as shown by the equivalent immune responses in NFκB-luciferase reporter macrophages to both PDX- and PTX-conjugated bacteria (FIG. 4C).

In these experiments, a control was included to assess whether attachment of these adjuvants to the cell surface was due to covalent, click-dependent ligation, or simply due to adsorption. To achieve this, the click reaction was performed under normal conditions (with alkynyl adjuvant, copper, ascorbic acid, TGTA, and aminoguanidine), except that the S. aureus cells were not treated with AAA-NHS to install azides at the cell surface. Therefore, any association of adjuvant with these cells would be due to nonspecific sticking. Using this control, as shown in FIG. 4C, it was found that adjuvant adsorption elicited no increase in immuno-stimulation over untreated S. aureus alone, demonstrating that the effects of conjugated PDX and PTX are attributable to click-mediated attachment rather than non-specific adherence.

Having established that a cleavable linker is not essential for the efficacy of the conjugatable adjuvant, it was sought to improve the solubility of the molecule by installing a polyethylene glycol (PEG) linker instead. Improving compound solubility was an important goal for two reasons. First, it was important to ensure that the conjugate—already relatively non-polar due to the hydrophobic xyliquimod moiety—would be soluble in aqueous solution at the 1 mM concentration necessary for sortase-mediated labeling. Second, improving solubility would help to reduce non-specific adherence of the conjugate to the surface of S. aureus, so that labeling of bacteria with the compound would predominantly represent sortase-mediated incorporation rather than sticking.

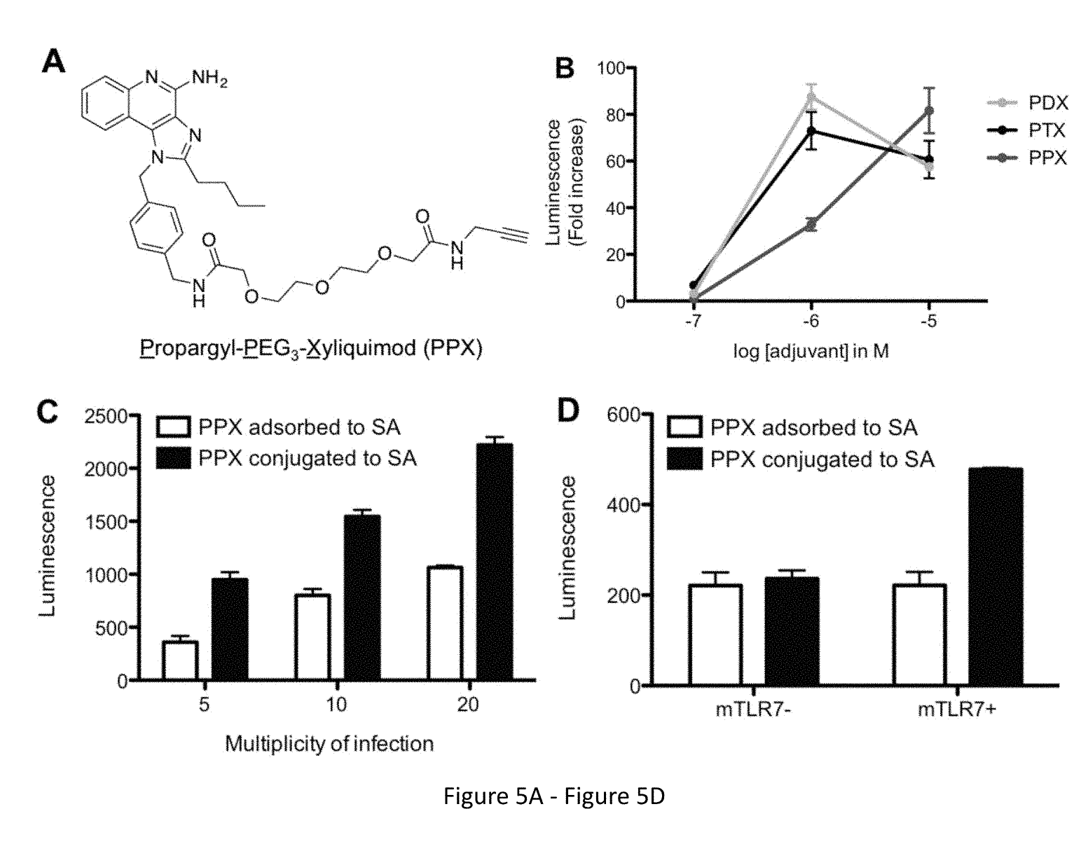

To confirm that a PEG-linked xyliquimod derivative would retain activity when conjugated to S. aureus, the simplified, “two-step” approach was again employed to attach the adjuvant to the cell surface. Although the activity of Propargyl-PEG3-Xyliquimod (PPX, FIG. 5A) in soluble form was slightly lower than PDX and PTX (FIG. 5B), it significantly potentiated immune responses to S. aureus when conjugated to its surface (FIG. 5C). Furthermore, this activation occurred in a TLR7-dependent manner, as shown by the mTLR7-HEK-NFκB-Luc reporter cell line assay (FIG. 5D).

Attempts to Conjugate Xyliquimod to S. aureus Via Sortase

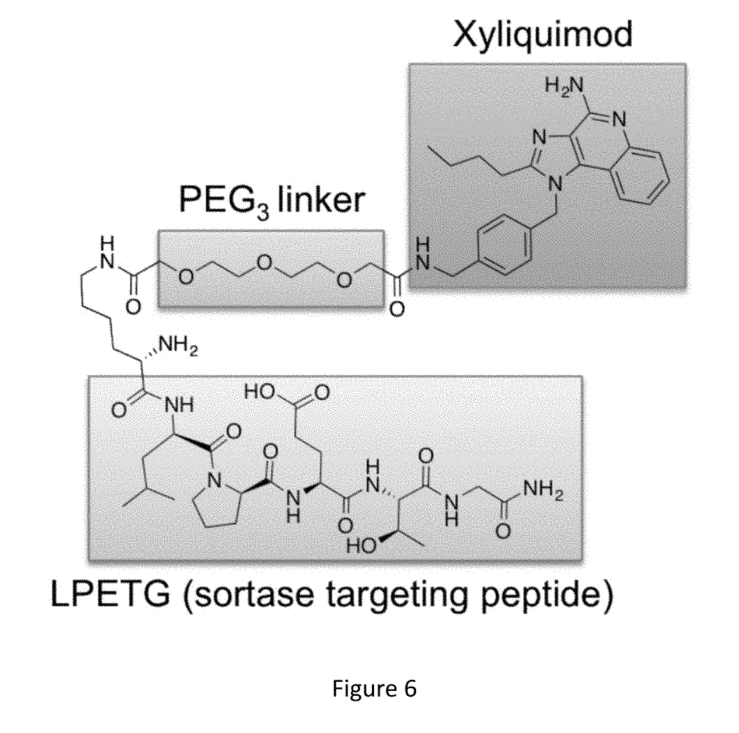

Having confirmed the efficacy of the PEG linker strategy, sortase-mediated installation of xyliquimod was investigated, and the Sortase-targeted-PEG3-Xyliquimod conjugate, SPX, was synthesized. (FIG. 6).

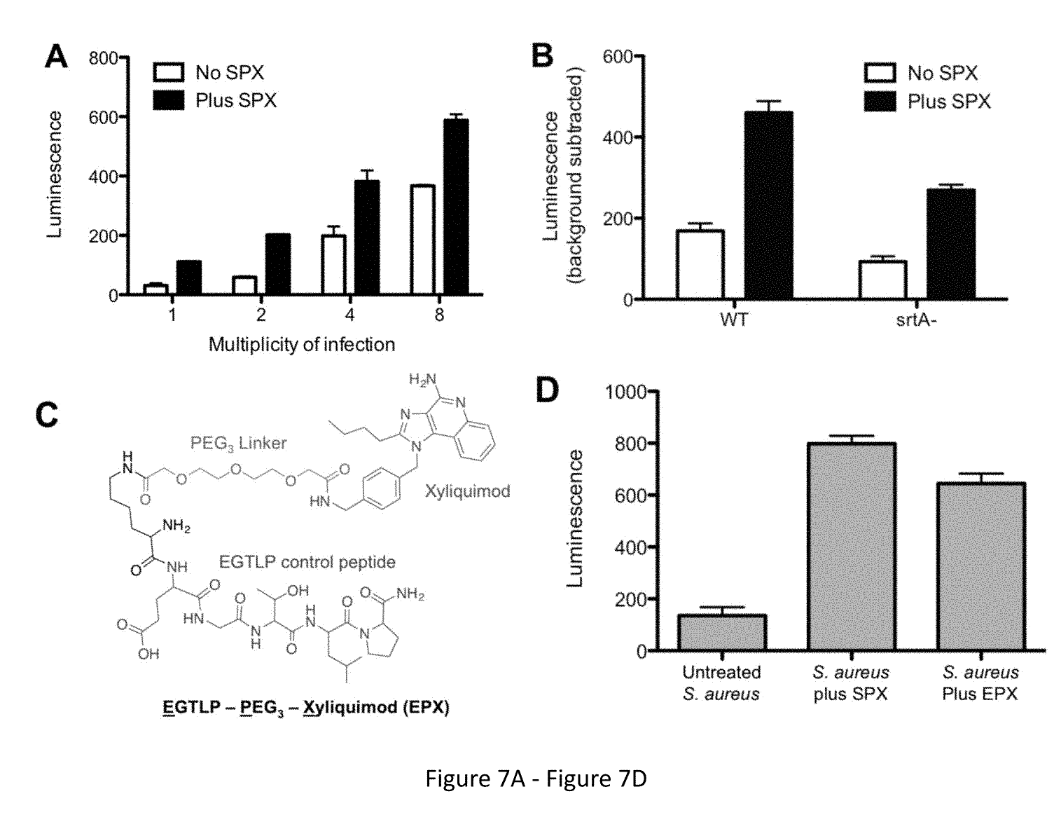

Initial testing with SPX was encouraging, as substantial immuno-potentiation was observed in RAW-NFκB-sLuc macrophages upon attachment of the compound to S. aureus (FIG. 7A). However, a comparison of immune responses to SPX-treated WT and sortase A-negative strains showed a significant SPX-dependent response to the AsrtA mutant, suggesting that the adjuvant was simply sticking to the surface of S. aureus in a sortase-independent manner (FIG. 7B). To explore this possibility further, an isomeric control compound containing the scrambled peptide, EGTLP was synthesized (EPX, FIG. 7C). Since this sequence is not recognized by sortase, any elicitation of immune responses by EPX-treated S. aureus would be attributable to non-specific adherence of peptide to the cell surface. In line with the AsrtA mutant studies (FIG. 7B), S. aureus treated with EPX generated a similar response to bacteria treated with SPX, demonstrating that xyliquimod-peptide conjugates do indeed stick to the surface of S. aureus in a sortase-independent manner (FIG. 7D).

As mentioned above, polar compounds tend to stick less avidly to the staphylococcal envelope than non-polar molecules. Therefore, to decrease non-specific adherence of SPX, it was sought to add a highly polar moiety to the compound. It was hypothesized that the attachment of fluorophore Alexa Fluor 488, which possesses two negatively charged sulfonic acid groups as well as a negatively charged carboxylic acid, would vastly improve the overall polarity of SPX. Its fluorescence would be a further advantage, as it would allow quantification of peptide labeling, providing a means to directly assess whether the adjuvant is incorporated via sortase or merely sticking.

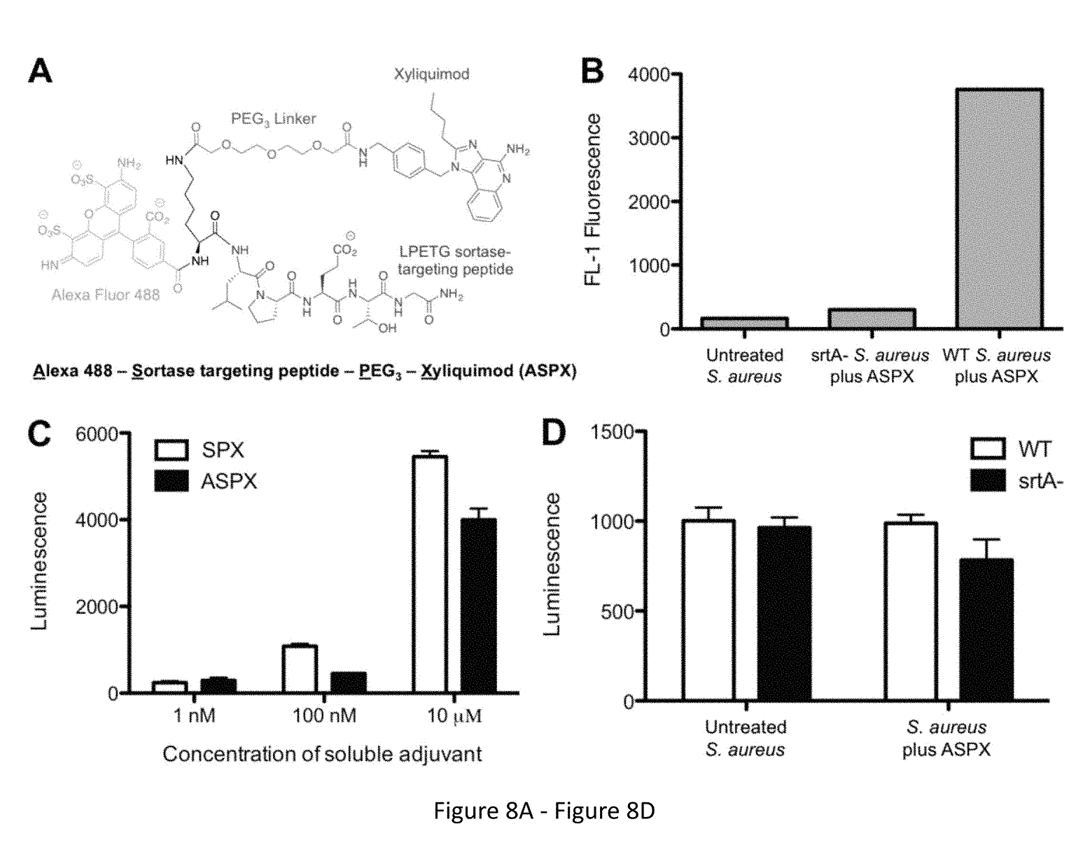

As hypothesized, the Alexa 488-SPX conjugate (ASPX, FIG. 8A) labeled S. aureus efficiently and showed negligible sortase-independent adherence to the cell envelope (FIG. 8B). Encouragingly, ASPX also proved immunologically active in soluble form (FIG. 8C). However, when conjugated to S. aureus, it failed to produce any detectable immune response (FIG. 8D). While not wishing to be bound to any particular theory, there may be several explanations for this failure. First, it has been shown that only ˜8000 molecules are incorporated into the cell wall of S. aureus using the sortase labeling system. In contrast, the two-step “click” labeling method for conjugating PPX, which produced robust immuno-stimulation, installs around 1,000,000 molecules per cell. Therefore, ASPX conjugation may simply fail to deliver an adequate dose of adjuvant to the macrophage to generate appreciable immune activation.

A second possible reason for ASPX's inefficacy is that cell-wall bound xyliquimod moieties may be inaccessible to TLR7 due to the barrier imposed by wall teichoic acids (WTA) at the staphylococcal cell surface. This theory fits well with fact that WTAs prevent binding of proteins to epitopes present within the cell wall of S. aureus. PPX molecules, meanwhile, are likely attached to these very WTA (via azide-modified D-alanine residues), and the resulting surface-exposure may well contribute to their efficacy. Of course, if the cell wall could be efficiently degraded by phagosomal hydrolases, then active xyliquimod derivatives attached to PG fragments would likely be released, leading to immune activation regardless of WTAs. However, the lysozyme resistance of S. aureus peptidoglycan likely prevents this from occurring. Thus, ASPX-delivered xyliquimod is likely buried within an indigestible cell wall, and obscured from detection due to the WTA layer at the cell surface. Given the fact that tunicamycin treatment of WT strains or use of a TagO mutant strain can reveal cell-wall bound epitopes through removal of WTA, it would be worth testing whether ASPX-modified cells are able to activate TLR7 after removal of WTA. Furthermore, based on the fact that that Oacetylation of N-acetylmuramic acid residues (mediated by oatA) is essential for lysozyme resistance, it may possible to achieve TLR7 activation with ASPX-modified ΔoatA S. aureus.

Finally, before leaving the sortase-based xyliquimod incorporation approach behind altogether, it was tested it in a more physiologically relevant experimental system. To this end, stimulations in murine bone marrow-derived macrophages (BMM) were performed and the resulting immune responses were assessed by quantifying expression of IL-1β, a pro-inflammatory cytokine transcriptionally regulated by NFκB. Besides more accurately recapitulating the physiological response to an immune stimulus than the RAW-NFκB-sLuc assay, the primary macrophage model offered two additional advantages over reporter cell lines. First, the dynamic range of qPCR analysis is far greater than that of the luciferase assay, so smaller differences in immune activation can be detected more readily. Second, because congenic TLR7KO cells were available, the role of TLR7 in xyliquimod-stimulated immune responses could be directly established without using the highly artificial mTLR7-HEK—NFκB-Luc reporter cell line, which is neither phagocytic nor even immune in lineage.

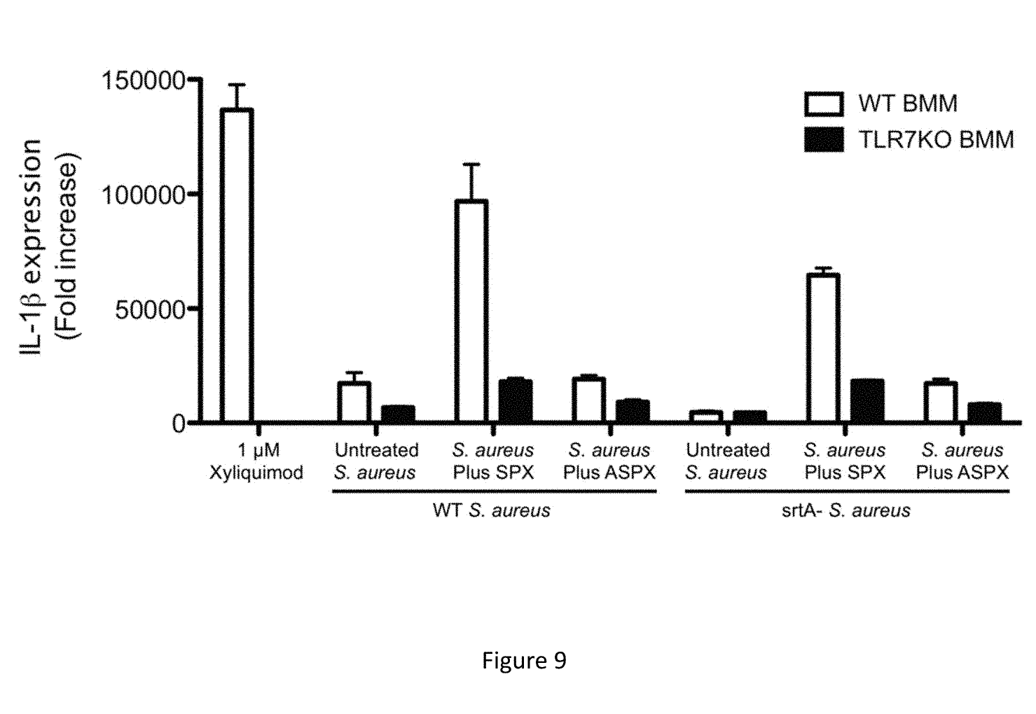

The results generated upon stimulation of BMMs are shown in FIG. 9. The first observation that emerged was that soluble xyliquimod is active in WT and not TLR7KO macrophages, confirming that the compound is a specific ligand for TLR7. Stimulations with modified bacteria, meanwhile, reflected many of the trends seen in earlier experiments. As before, an increased immune response was observed upon treatment of WT S. aureus with SPX. However, SPX-treated ΔsrtA bacteria elicited a similar response, confirming that the compound's effects are largely sortase-independent, attributable instead to sticking to the bacterial surface. Also consistent with previous results, ASPX treatment—which results in clean, sortase-mediated incorporation of xyliquimod into the cell wall—produced no changes in IL-1β expression. Together, these results substantiated the conclusions made using the RAW-NFκB-sLuc system, strongly suggesting that an alternative to the sortase labeling approach should be pursued.

Before proceeding, one final observation from these data is worth noting. WT S. aureus consistently generated stronger immune responses than the ΔsrtA mutant—a trend which was also observed in RAW-NFκB-sLuc experiments as well (FIG. 7B). Furthermore, it appeared that the increased immunogenicity of sortase-expressing S. aureus seemed to be dependent on TLR7, given the lack of immune response to WT S. aureus in the TLR7KO BMMs. While not wishing to be bound to any particular theory, the simplest explanation for this unexpected finding is that some sortase-incorporated protein is able to serve as a ligand for TLR7, but this seems unlikely given the receptor's natural affinity for single-stranded RNA. Instead, the TLR7 agonist may be RNA in the cytosol of S. aureus that somehow survives the heat-killing process, which is known to induce RNA degradation. In this case, the WT strain may simply possess a sortase-incorporated protein that promotes phagocytosis by macrophages, thus increasing delivery of a passenger TLR7 ligand.

Xyliquimod Conjugation to S. aureus Via Copper-Catalyzed Click Chemistry Greatly Enhances its Immunogenicity

Although attempts to install xyliquimod on the staphylococcal cell surface via sortase ultimately proved unsuccessful, they did yield a compound, PPX, which showed excellent immuno-stimulatory activity when attached to S. aureus. Initially, the changes of success in employing the two-step labeling system necessary for PPX conjugation—i.e. treatment with AAA-NHS to coat cells with azides followed by click ligation of PPX to the azide handles—were uncertain because of the theoretical risk of compromising antigenicity of surface proteins through acylation of their free amines with AAA-NHS. However killing pathogens with acylating compounds like β-propiolactone actually preserves antigenicity more effectively than any other means of inactivation reported, including treatment with heat, ethanol, chloroform or trichloroethylene (Lu et al., 2010, Clin Vaccine Immunol 17(6):1005-12). Thus, it was decided to proceed with PPX as the conjugatable adjuvant.

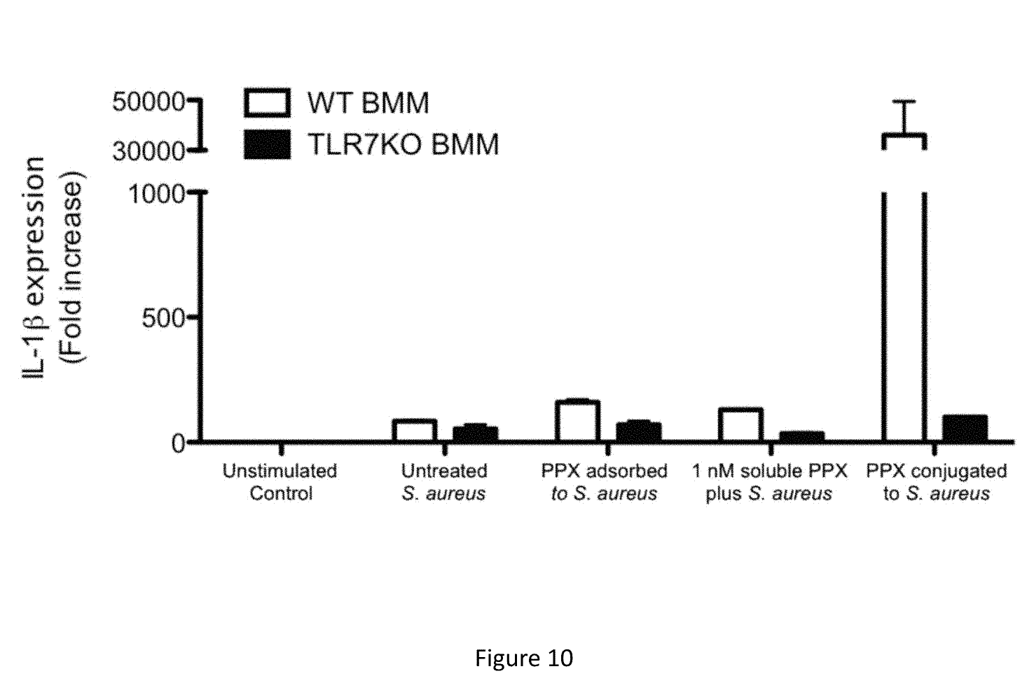

First, it was sought to characterize the effects of PPX conjugation in primary BMM as described in the previous section. Remarkably, stimulation with PPX-conjugated cells elicited a >200 fold increase in IL-1β expression compared with untreated bacteria alone (FIG. 10). While not wishing to be bound to any particular theory, it is possible that this dramatic effect is a consequence of synergy between the attached xyliquimod and the intrinsic PAMPs present within S. aureus. Alternatively, it may simply be due to the extraordinary potency of this chemical adjuvant, which far outstrips the cumulative immuno-stimulatory effects of all of the staphylococcal PAMPs together.

Importantly, it was observed that the PPX-induced increase in BMM activation was entirely abrogated in TLR7KO macrophages, showing that adjuvanticity of PPX conjugation is attributable to TLR7, as originally hypothesized. In addition, it was shown that PPX was in fact conjugated to S. aureus, rather than sticking nonspecifically, as PPX adsorbed to bacteria at the same concentration used for the click reaction had negligible immuno-stimulatory effects.

Finally, it was sought to demonstrate the impact of adjuvant conjugation, which allows targeted delivery of xyliquimod to the TLR7-expressing phagosomal compartment. To do this, a control was included in which unmodified S. aureus was co-administered with an amount of PPX equimolar to that attached to the surface of PPX-conjugated S. aureus. This amount was estimated using the following approach. First, the number of PPX molecules attached to each individual cell was approximated by conjugating a fluorescent alkynyl probe (structurally analogous to PPX) to AAA-NHS-treated S. aureus. The fluorescence intensity of these cells was then plotted on a standard curve obtained using reference microspheres containing known amounts of fluorescein molecules. This process yielded an approximate value of 1,000,000 alkynyl fluorescein molecules per cell. Next, assuming a similar number of PPX molecules are conjugated to bacteria through the click reaction, this value was used, the number of bacterial cells added to each experimental well, and the well volume to calculate an effective concentration of 800 pM of PPX in BMM stimulation experiments. As shown in FIG. 5B, this concentration is several orders of magnitude below the EC₅₀ of PPX, and is therefore highly unlikely to affect immune responses. Indeed, when co-administered with S. aureus, 1 nM PPX had no detectable effects on BMM activation. These results provide dramatic evidence for the efficacy of adjuvant conjugation and targeted delivery—to achieve a level of immuno-potentiation similar to conjugated PPX would likely require a >100-fold higher concentration of soluble adjuvant.

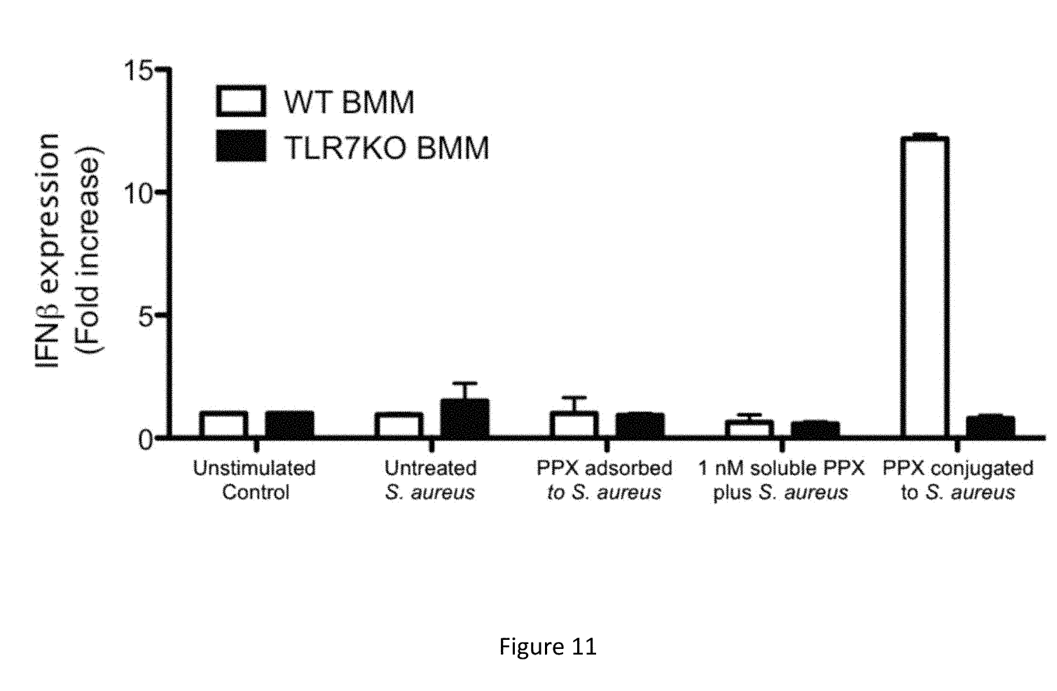

Next, it was determined if PPX conjugation would elicit production of type I IFNs. As described above, these cytokines (IFNα and IFNβ) have been associated with the innate immune response to live bacteria, which are known to stimulate stronger immune responses than their killed counterparts. Excitingly, as shown in FIG. 11, attachment of PPX did induce IFNβ expression in BMM, in a TLR7- and conjugation-dependent manner. It should be noted that the relatively smaller induction of IFNβ (˜12-fold) compared to IL-1β (˜40,000-fold) reflects the lower dynamic range of IFNβ gene expression rather than poor stimulation of the IFNβ pathway. Also noteworthy is that native S. aureus elicited no detectable type I IFN response, as has been observed previously (Kaplan et al., 2012, J Immunol 189(9):4537-45). Thus, through conjugation of xyliquimod, killed S. aureus is endowed with a completely novel immunological property, one known to promote Th1 immunity (Tough et al., 2012, Immunol Cell Biol 90(5):492-7), and one that may help to generate the robust adaptive responses characteristic of live vaccines.

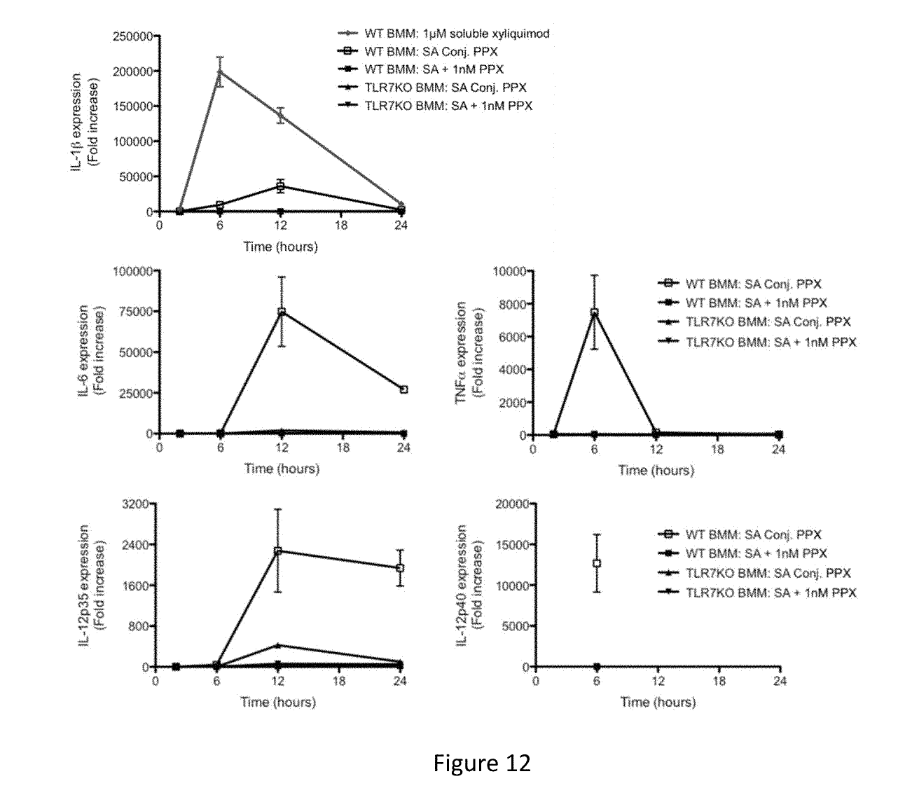

To more fully characterize the effect of PPX conjugation, the expression of several additional cytokines over time was analyzed in BMMs. First, an interesting phenomenon was observed in which the peak transcriptional response to PPX-conjugated S. aureus versus appeared substantially later than the peak response to soluble xyliquimod (FIG. 12). This delay may be attributable to the time required to degrade the cell envelope components to which PPX is attached (e.g. WTA) in order to release TLR7-active derivatives from PPX-conjugated S. aureus.

In FIG. 12, it is demonstrated that PPX conjugation stimulates TLR7- and conjugation-dependent expression of additional pro-inflammatory genes (IL-6 and TNFα), and of genes for both subunits of IL-12, a critical cytokine in the generation of Th1 immune responses. Notably, an increase in expression of IL-23 was not observed, the primary cytokine involved in Th17 differentiation (data not shown). All together, these data show that PPX conjugation promotes robust activation of pro-inflammatory, type I IFN, and Th1-promoting immune responses from primary macrophages in a TLR7-dependent manner. In addition, the physical attachment of adjuvant to pathogen—a concept never before explored in vaccine design—was shown to dramatically decrease the amount of adjuvant necessary to elicit immune activation.

Cyclooctynyl Derivatives Enable Xyliquimod Conjugation to S. aureus Via Copper-Free Click Chemistry

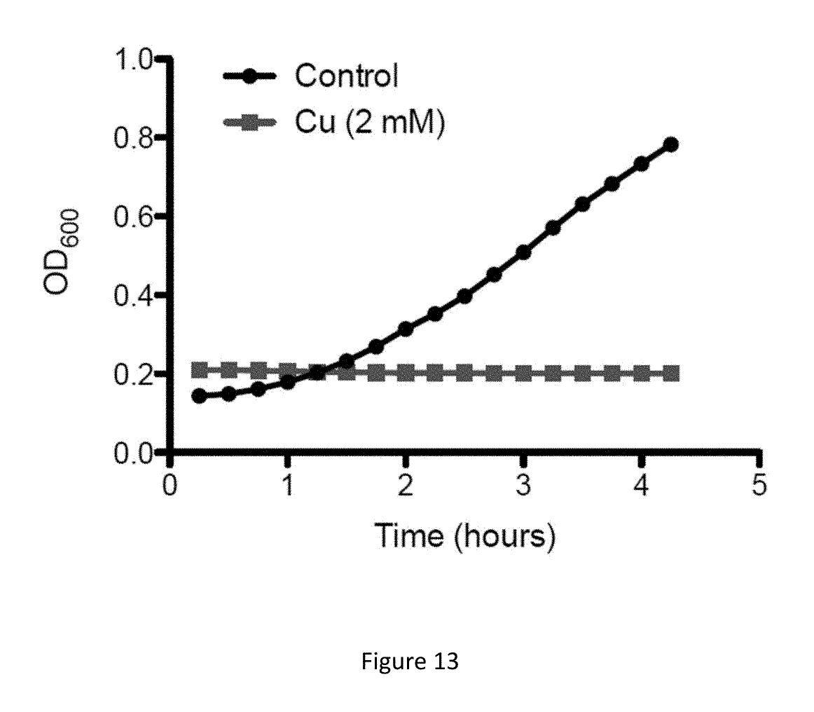

Given the efficacy of PPX-conjugated S. aureus in vitro, proceeding at this point to testing in murine vaccine models was considered. However, it was possible that the 2 mM copper concentration required for click conjugation might have an adverse effect on the antigenicity of surface proteins. As shown in FIG. 13, this concentration did completely inhibit S. aureus growth, but more importantly, the reactive oxygen species generated from redox cycling of copper ions between Cu(I) and Cu(II) during the click reaction may lead to protein oxidation, thus compromising antigenic structure. Although this was only a theoretical concern, it seemed an unnecessary risk given the existence of technology that allows copper-free click ligation of cyclooctynyl derivatives to azides (Jewett, 2010, Chem Soc Rev 39(4):1272-9).

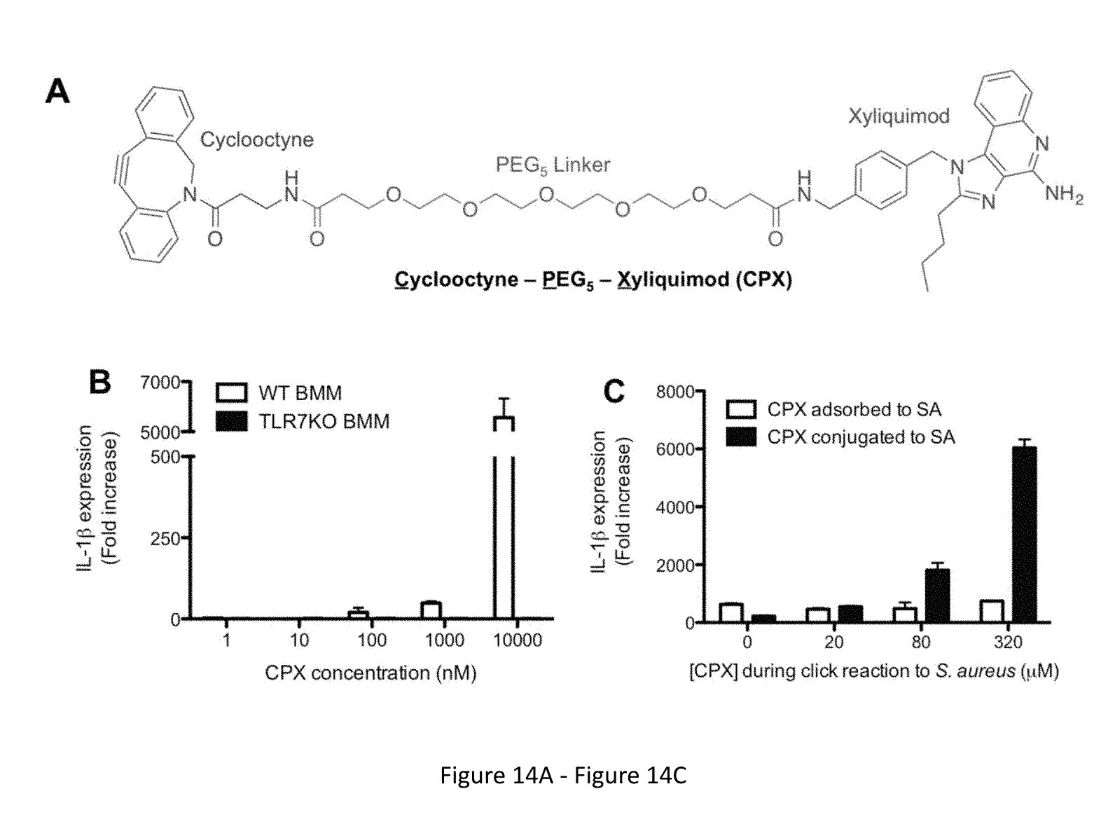

Therefore, a xyliquimod derivative possessing a strained cyclooctyne moiety was designed and synthesized that would permit copper-free ligation to AAA-NHS-treated S. aureus (CPX, FIG. 14A). To start, the soluble form of CPX robustly stimulated IL-1β expression in BMM in a TLR7-dependent manner (FIG. 14B). Its immuno-stimulatory potential upon attachment to S. aureus was assessed. Although CPX-conjugated bacteria did induce IL-1β expression in BMM, it was noted during these experiments that the compound was poorly soluble at concentrations above ˜100 μM (FIG. 14C). Since efficacy was clearly related to the concentration of CPX present during the conjugation reaction (FIG. 14C), it was necessary to modify the compound in order to increase its solubility.

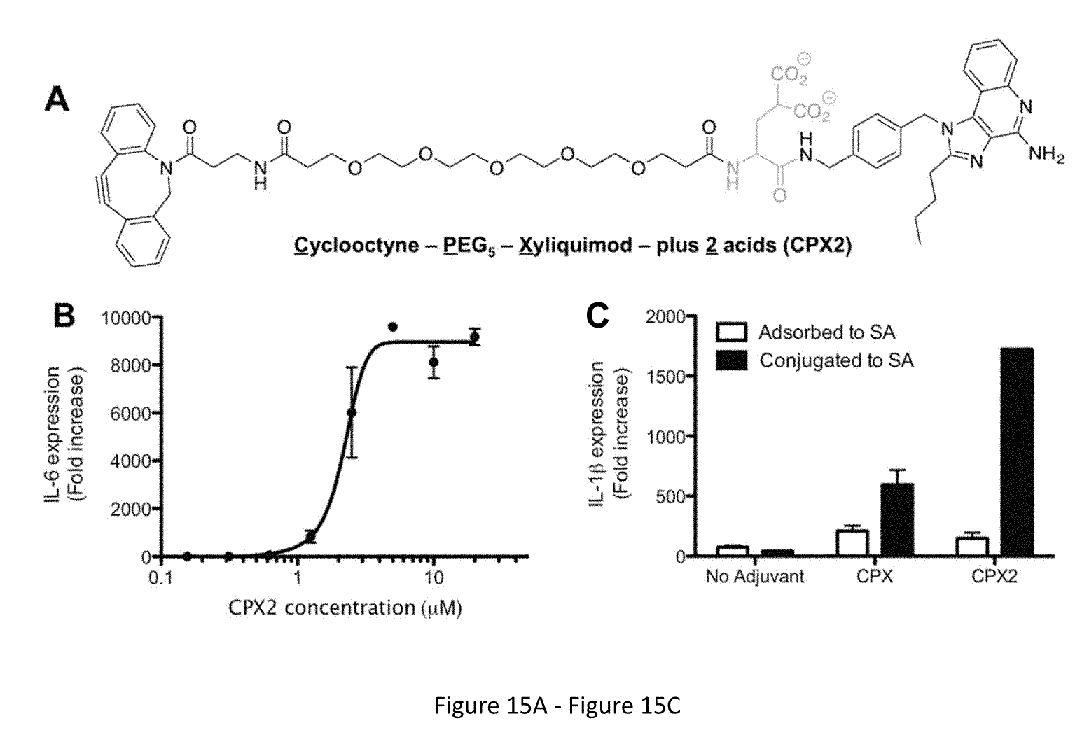

To this end, a CPX derivative containing two carboxylic acid moieties was synthesized, which provided two negative charges at the neutral pH of the reaction (CPX2, FIG. 15A). As hypothesized, this modification greatly increased the aqueous solubility of the compound, and did not compromise efficacy (FIG. 15B). When compared directly to CPX (click reaction performed at 100 nM), CPX2 conjugation elicited significantly stronger immune responses, and also showed reduced non-specific sticking (FIG. 15C).

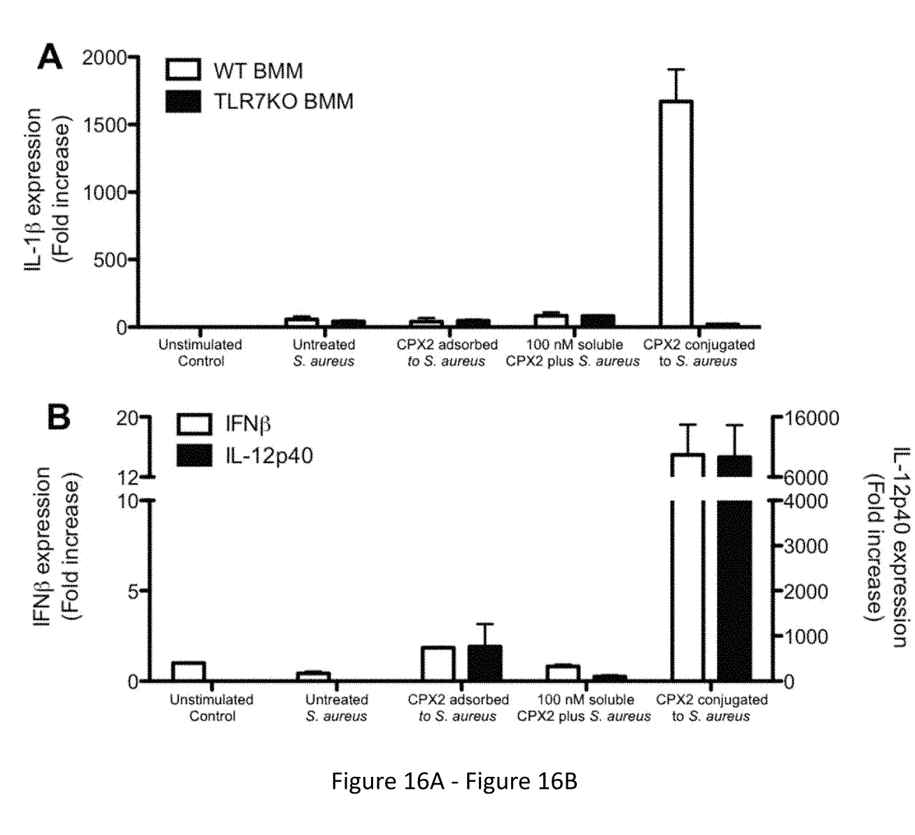

Having finalized the structure of the “clickable adjuvant”, CPX2, its immuno-stimulatory properties were characterized in primary immune cells. Similar to PPX, CPX2 elicited strong up-regulation of pro-inflammatory (IL-1β), type I IFN (IFNβ), and Th1-promoting (IL-12b) genes in BMM (FIGS. 16A and 16B). The adjuvanticity of CPX2 was also shown to be dependent on TLR7 and click-mediated conjugation to S. aureus (FIG. 16A).

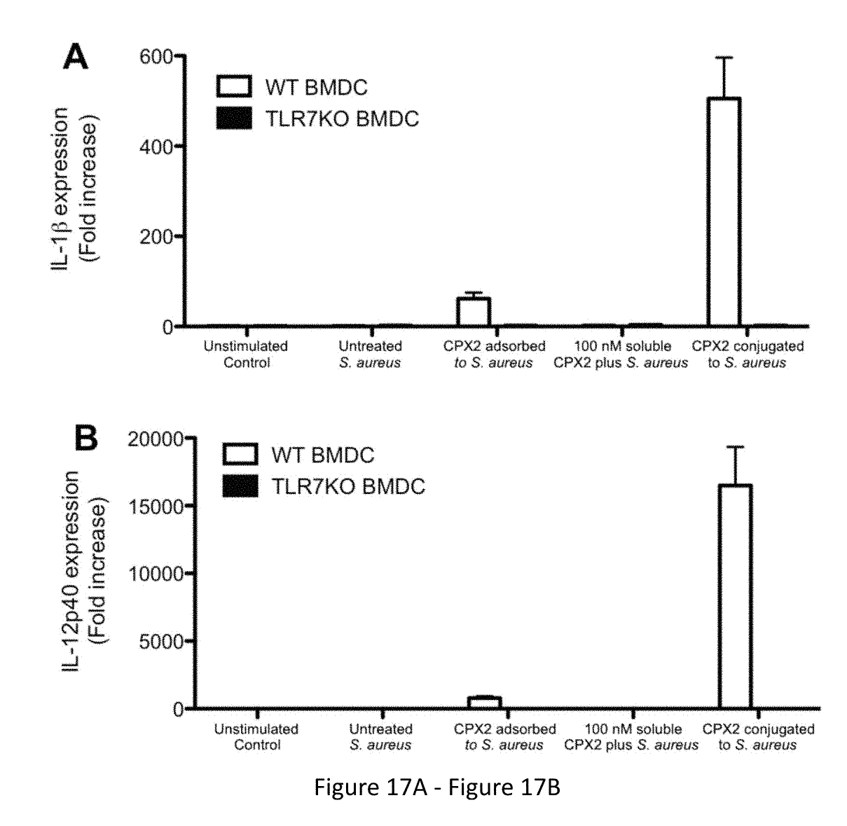

In order to argue more confidently for its potential as a vaccine, it was assessed whether CPX2-conjugated S. aureus could activate dendritic cells—the principal APCs involved with the generation of adaptive immune responses. To this end, stimulations in bone marrow-derived dendritic cells (BMDC) were performed and cytokine gene expression was assessed. As shown in FIG. 17, similar changes as in BMM were observed: pro-inflammatory and Th1-promoting genes were both strongly induced in both a TLR7- and conjugation-dependent manner.

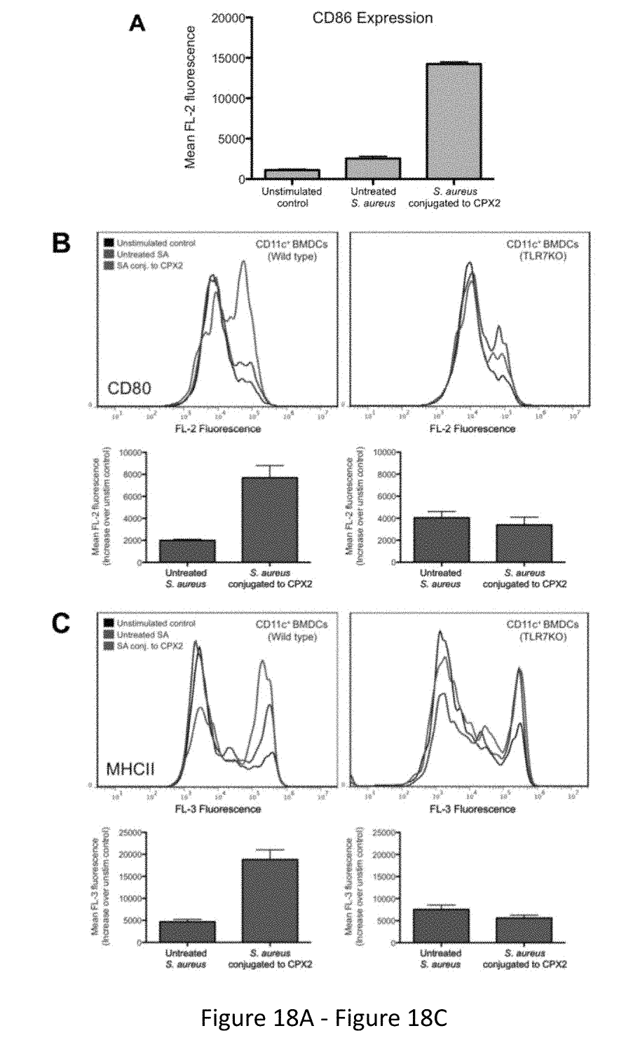

Finally, the effects of CPX2 conjugation on expression of maturation markers in BMDCs were evaluated. As shown in FIG. 2, the CD4+ T cell-stimulating potential of a dendritic cell is dependent not only on the cytokines that were analyzed (signal 3), but also on cell surface expression of MHC-II (signal 1) and co-stimulatory markers such as CD80 and CD86 (signal 2). Therefore flow cytometric quantification of these maturation markers was performed on BMDCs stimulated with CPX2-conjugated S. aureus. As shown in FIG. 18A, a dramatic rise in CD86 expression in response to CPX2-modified bacteria, and only a slight rise upon addition of untreated S. aureus was observed. Analysis of CD80 and MHCII in WT and TLR7KO BMDCs revealed a TLR7- and CPX2-dependent increase in both markers (FIG. 18B). Taken together with the gene expression studies shown above, these experiments provide strong evidence that CPX2-conjugated S. aureus potently activates dendritic cells, promoting antigen presentation, co-stimulation, and synthesis of cytokines that trigger adaptive immune responses.

The data described herein introduce a novel class of compounds that allow physical attachment of immuno-stimulatory molecules to intact bacteria, which are termed herein as “clickable adjuvants.” The prototype compound characterized here is CPX2, a polar, cyclooctyne-containing imidazoquinoline derivative. Through simple incubation with azide-coated S. aureus, CPX2 is robustly conjugated to the cell surface, and induces potent activation of APCs in a TLR7-dependent manner. In addition to up-regulating MHCII, co-stimulatory markers, pro-inflammatory genes, and Th1-promoting cytokines, CPX2 also triggers expression of type I IFNs—a response usually reserved for live bacteria.

The possibility that induction of this “live bacterial cytokine signature” will promote the stronger adaptive immune responses that have been associated with live vaccines in the past is intriguing. Further testing of this theory would require an in vivo model that reveals vaccine-induced protection, but once that is established, the role of type I interferons in vaccine efficacy can easily be assessed by performing vaccine studies in the interferon receptor knockout mouse (IFNAR^{-/-}).

Physical coupling of CPX2 to S. aureus allows highly efficient delivery of adjuvant to APCs, greatly reducing the amount of compound necessary for achieving robust immune responses. While not wishing to be bound to any particular theory, this novel approach may help to solve one of the critical problems facing vaccine research at present: how to formulate antigen with adjuvant in a safe and effective manner. Again, however, definitive demonstration of the adjuvant-sparing effect of conjugation would require a functional in vivo vaccine model.

As discussed above, it was hypothesized that attaching IMQ to the staphylococcal surface per se would be advantageous, as the surface-exposed adjuvant would likely engage TLR7 soon after phagocytosis, leading to a phenomenon known as phagosome-intrinsic PRR signaling. Such signaling is known to augment phagosomal maturation, fusion with lysosomes, and export of MHCII:peptide complexes to the plasma membrane. As noted earlier, WWII presentation is inefficient in APCs treated with S. aureus, perhaps due to the poor accessibility of PAMPs buried within a degradation resistant cell wall. Superficial exposure of adjuvant, therefore, may provide a signal that triggers more efficient presentation of S. aureus antigens to T cells. As discussed above, this hypothesis may be tested using the ASPX molecule.

Chemically adjuvanted whole cell vaccination holds great promise as a vaccine design moving forward, for several reasons in addition to those mentioned above. First, the use of whole S. aureus, which naturally expresses a full complement of staphylococcal antigens, offers hope that the vaccine may provide broad protection against S. aureus infection, as has recently been shown with whole cell vaccines against both bacterial and fungal pathogens (Chen et al., 2011, Immunity 35(5):9971009; Malley and Anderson, 2012, PNAS 109(10):3623-7; Wuthrich et al., 2011, J Clin Invest 121(2):554-68; Wuthrich et al., 2011, J Immunol 187(3):1421-31). Second, the use of intact cells (rather than soluble antigen) allows delivery of antigen via phagocytosis, which has been shown to increase immunogenicity per se, and may even enable immunization through mucosal routes. This mode of vaccination offers several advantages over parenteral delivery, including improved safety of administration, and protection against not only invasive, but initial infection as well.

Finally, it should be recognized that the novel chemical methodology for IMQ conjugation to cell surfaces is compatible with virtually any culturable pathogen, including bacteria and fungi. Thus, in the future, as additional clickable adjuvants become available, a modular approach for vaccine generation is envisioned in which one simply identifies a target pathogen and then selects the adjuvant that elicits immune responses most appropriate for defense against the etiologic agent. This facile, inexpensive methodology thus represents one step closer towards the goal of rational vaccine design, towards an answer to infectious diseases such as S. aureus, which represent an urgent and growing clinical problem in the U.S. and abroad.

The materials and methods are now described.

Bacterial Culture and Chemical Modification

WT S. aureus Newman was grown routinely in Luria Bertani (LB) broth at 37° C. with agitation at 200 RPM. For sortase peptide labeling experiments, liquid cultures were inoculated with single colonies, grown overnight to stationary phase, and then diluted 1:100 in LB containing 1 mM of the indicated peptide. Cultures were then grown overnight (12-16 hours), washed with PBS, killed by heating at 65° C., washed again, re-suspended in PBS, and counted by flow cytometry. For click labeling experiments, liquid cultures were inoculated with single colonies, grown overnight to stationary phase, washed with PBS, killed by heating at 65° C. for 30 minutes, and washed again. Cells were then suspended in basified PBS (pH 9), and azidoacetic acid NHS ester (AAA-NHS) was added from a DMSO stock to reach a final concentration of 2.5 mM. The reaction was allowed to proceed for 20 minutes before cells were washed with PBS. Copper-catalyzed “click” conjugation of terminal alkyne-functionalized xyliquimod derivatives was performed according to literature precedent with slight modifications. First, CuSO4 (2 mM) and tris[(1-glucosyl-1H-1,2,3-triazol-4-yl)methyl]amine (TGTA, 4 mM) were incubated together in PBS at 37° C. for 4 hours. To this mixture, aminoguanidine (4 mM), alkynyl xyliquimod (1 mM) and ascorbic acid (5 mM) were added in order. AAA-NHS-treated S. aureus (coated with azide moieties) was then re-suspended in the click reaction mixture for 15 minutes at 37° C. before washing in PBS and counting by flow cytometry. Copper-free “click” conjugation of cyclooctyne-functionalized xyliquimod derivatives was performed by incubating AAA-NHS-treated S. aureus with adjuvant (at 500 μM) in PBS for 30 minutes at room temperature. Cells were then washed, re-suspended in PBS, and counted by flow cytometry. To obtain live S. aureus for lethal intravenous challenge in vaccine experiments, liquid cultures were inoculated with single colonies, grown overnight to stationary phase, washed, and re-suspended in PBS. Cell counts were assessed by measuring absorbance at OD₆₀₀.

Description of Luciferase Reporter Cell Lines

A RAW 264.7 macrophage-like cell line stably transfected with a construct that generates secreted metridia luciferase upon activation of NFκB was constructed previously by Tina Wang (Spiegel laboratory). This cell line was cultured in G418-containing DMEM media to maintain expression of the transgene. An mTLR7-NFκB-Luc HEK293 cell line was made from a NFκB-Luc HEK293 cell line stably transfected with a construct that generates cytoplasmic firefly luciferase upon activation of NFκB, which was acquired from the laboratory of Ruslan Medzhitov. The NFκB-Luc construct was maintained using G418 as a selection agent and grown in DMEM media. A plasmid expressing an HA-tagged version of the mouse TLR7 (mTLR7) gene was acquired from Origene (Cat. #: pUNO1-mTLR7-HA3x), and stably transfected into the NFκB-Luc HEK293 cell line using the antibiotic blasticidin as a selection agent. A control cell line was constructed by stably transfecting an empty pUNO1 vector into the NFκB-Luc HEK293 cell line. After selection with blastocidin (40 μg/mL) for one week, cells were split and transferred to 10 cm dishes at low confluence. Colonies were picked using sterile pipette tips and transferred to 96 well plates without trypsinization. After reaching confluence, cells were split and transferred to larger wells for testing by functional assay. The two clones showing optimal functional responses were amplified and stocks were stored in liquid nitrogen.

Stimulation of RAW-NFκB-sLuc Cell Line

Soluble compounds and bacterial samples (with and without chemical modifications) were added to confluent RAWNFκB-sLuc cells grown in 48 well plates, and incubated for indicated lengths of time. 30 μl of media from each sample was transferred to a well of a 96 well plate. 70 μl of coelenterazine (Santa Cruz) solution in PBS was added to each well to give a final coelenterazine concentration of 150 μM. Luminescence was immediately measured on a microplate reader (Tecan, Reading, UK).

Stimulation of mTLR7-HEK—NFκB-Luc Cell Line

Soluble compounds and bacterial samples (with and without chemical modifications) were added to confluent mTLR7-HEK—NFκB-Luc cells plated in 96 well plates (as well as to the empty vector-transfected control line) and incubated for indicated lengths of time. After stimulation, supernatant was removed and lysis buffer was added (Promega, Madison, Wis.). Firefly luciferase substrate solution (Promega) was then added according to the manufacturer's instructions. Luminescence was measured using a microplate reader (Tecan) shortly after addition of substrate.

Stimulation of Primary Immune Cells with Adjuvanted S. aureus

BMMs and BMDCs were prepared from bone marrow progenitor cells harvested from C57BL/6 mice and cultured for 6 days on Petri dishes in RPMI-1640 media supplemented with macrophage colony-stimulating factor (BMM) or granulocyte macrophage colonystimulating factor (BMDC). Cells were fed with fresh media on day 4 and were lifted (with 5 mM EDTA in the case of macrophages) and re-plated on 48-well tissue culturetreated dishes (200,000 cells per well) the day before stimulation. Immune cells were stimulated with S. aureus samples and soluble adjuvants for lengths of time as indicated.

qPCR Analysis of Cytokine Expression in Primary Immune Cells

After stimulation of BMDCs and BMMs, the supernatant was removed, cells were lysed, and RNA was isolated using the RNeasy kit (Qiagen, Germantown, Md.) according to manufacturer's instructions. cDNA was reverse-transcribed with an oligo(dT) primer using Moloney murine leukemia virus reverse transcriptase (MMLV RT). cDNA was analyzed in triplicate by qPCR amplification using SYBR Green Supermix (Life Technologies, Carlsbad, Calif.) on a Bio-Rad CFX96 Real-Time PCR Detection System (Bio-Rad, Hercules, Calif.). PCR amplification conditions were as follows: 95° C. (3 minutes) and 45 cycles of 95° C. (15 seconds) and 60° C. (45 seconds). Primer pairs (Table 1) were used to amplify mRNA-specific fragments, and unique products were tested by melt-curve analysis [95° C. (15 seconds), 60-95° C. (Δ5° C., 5 seconds)]. Relative expression was normalized to the ribosomal protein, L13a (Rp113a). Data are represented as the fold induction over unstimulated cells.

TABLE 1
Primers used in qPCR

| | Forward Primer | Reverse Primer |
|---|---|---|
| Gene | (5′ → 3′) | (5′ → 3′) |
| Il6 | AAGCCAGAGTCCTTC | TAAGGGTCTGCTTCT |
| | AGAGAGATA | CCCACAGGAGGTT |
| | (SEQ ID NO: 2) | (SEQ ID NO: 3) |
| Il12p35 | CACAAGAACGAGAGT | ACTTGAGGGAGAAGT |
| | TGCCTGGCTACTA | AGGAATGG |
| | (SEQ ID NO: 4) | (SEQ ID NO: 5) |
| Il12p40 | ATGAGAACTACAGCA | GAGAACCTGGGAGTA |
| | CCAGCTTC | GACAAGGT |
| | (SEQ ID NO 6:) | (SEQ ID NO: 7) |
| Tnfa | AAATTCGAGTGACAA | CTGCTGCTGGTGATC |
| | GCCTGTAG | CTCTTG |
| | (SEQ ID NO: 8) | (SEQ ID NO: 9) |
| IFNb | GCACTGGGTGGAATG | TTCTGAGGCATCAAC |
| | AGACTATTG | TGACAGGTC |
| | (SEQ ID NO: 10) | (SEQ ID NO: 11) |
| IL1b | CAGTTGTCTAATGGG | GCACCTTCTTTTCCT |
| | AACGTCA | TCATCTTT |
| | (SEQ ID NO: 12) | (SEQ ID NO: 13) |
| Rpl13a | GCTGCCGAAGATGGC | ACCACCACCTTCCGG |
| | GGAGG | CCCA |
| | (SEQ ID NO: 14) | (SEQ ID NO: 15) |

Analysis of Maturation Markers on Bone Marrow-Derived Dendritic Cells

BMDCs were stimulated with modified S. aureus at a multiplicity of infection (MOI) of 10 for 24 hours and then collected in 100 ul cold PBS containing 1:200 Fc block (rat antimouse CD16/CD32, cat. #553141, Becton Dickinson, Franklin Lakes, N.J.) by scraping. Cells were then centrifuged, and re-suspended in 100 ul FACS buffer (10% ultra-low IgG serum plus penicillin and streptomycin in PBS) containing 1:100 dilutions of antibody-conjugates: phycoerythrin (PE)-conjugated anti-mouse CD80 (clone 16-10A1); PEconjugated anti-mouse CD86 (clone P03); peridinin chlorophyll protein (PerCP)/Cy5.5 anti-mouse MHCII (I-A/I-E) (clone M5/114.15.2); allophycocyanin (APC) anti-mouse CD11c (clone N418). Cells were incubated on ice for 15 minutes, washed, re-suspended in FACS buffer, and analyzed by flow cytometry.

Flow Cytometry

For bacterial analysis, samples were analyzed on an Accuri C6 flow cytometer (BD, Franklin Lakes, N.J.) on medium speed fluidics with a minimum threshold of 40,000 FSC-H. Maximum FSC and SSC gates were set to exclude multi-cell aggregates (e.g. 50,000 FSC-A and 50,000 SSC-A). For assessment of surface marker expression in BMDCs, 10,000 events were measured on fast speed fluidics with a minimum threshold of 80,000 FSC-H. A polygonal plot was drawn around live BMDCs as defined by FSC-A and SSC-A, and analysis was performed on this population following color compensation. Traces were processed using FlowJo software (Ashland, Oreg.).

Quantification of Molecules Conjugated to S. aureus Via Click Chemistry

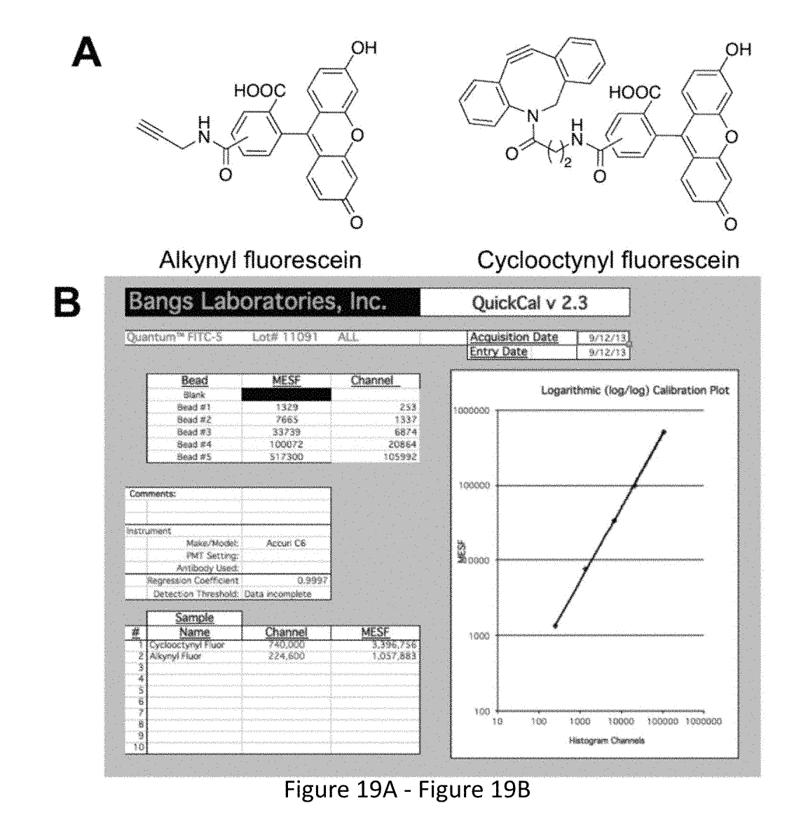

To approximate the number of molecules ligated to S. aureus by click chemistry, click reactions with alkynyl fluorescein were performed and cyclooctynyl fluorescein (Dibenzocyclooctyne-fluor 488, Sigma-Aldrich, St Louis, Mo.) under analogous conditions as those used to conjugate PPX and CPX2, respectively (FIG. 19A). Total cellular fluorescence were assessed by flow cytometry and plotted the value on a curve relating number of fluorescein molecules to fluorescence intensity (FIG. 19B), generated using the MESF FITC quantitation kit and QuickCal 2.3 according to manufacturer's protocols (555 Quantum FITC-5 MESF, Bangs Laboratories, Fishers, Ind.).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A cell comprising an immunomodulatory molecule bound to the cell surface.

2. The cell of claim 1, wherein the cell is selected from the group consisting of a bacterial cell and a fungal cell.

3. The cell of claim 2, wherein the bacterial cell is an inactivated bacterial cell.

4. The cell of claim 2, wherein the bacterial cell is a Staphylococcus sp. cell.

5. The cell of claim 5, wherein the Staphylococcus sp. cell is a Staphylococcus aureus cell.

6. The cell of claim 1, wherein the immunomodulatory molecule comprises an adjuvant group, a linker group and a targeting group.

7. The cell of claim 6, wherein the targeting group selected from the group consisting of a group capable of participating in a click-chemistry reaction and a group capable of participating in a sortase-mediated labeling reaction.

8. The cell of claim 7, wherein the group capable of participating in a click-chemistry reaction comprises a group selected from the group consisting of a terminal alkyne, a terminal azide, and a cyclooctyne group.

9. The cell of claim 6, wherein the group capable of participating in a sortase-mediated labeling reaction comprises a sortase targeting peptide.

10. The cell of claim 9, wherein the sortase targeting peptide comprises the amino acid sequence LPETG (SEQ ID NO:1).

11. The cell of claim 6, wherein the linker group bridges the adjuvant group and the targeting group.

12. The cell of claim 6, wherein the linker comprises a group selected from the group consisting of a disulfide linking group, a thioester group, and a polyethylene glycol (PEG) group.

13. The cell of claim 6, wherein the adjuvant comprises a group selected from the group consisting of a imidazoquinoline and a imidazoquinoline derivative.

14. The cell of claim 1, wherein the immunomodulatory molecule is selected from the group consisting of: 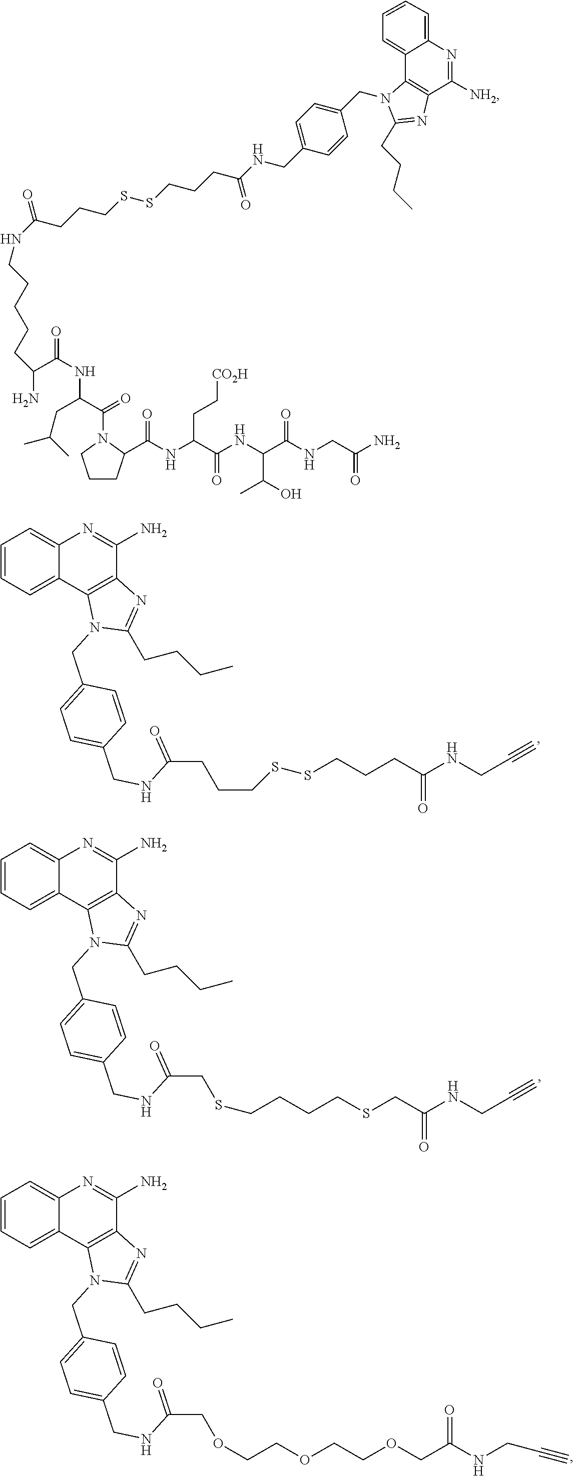 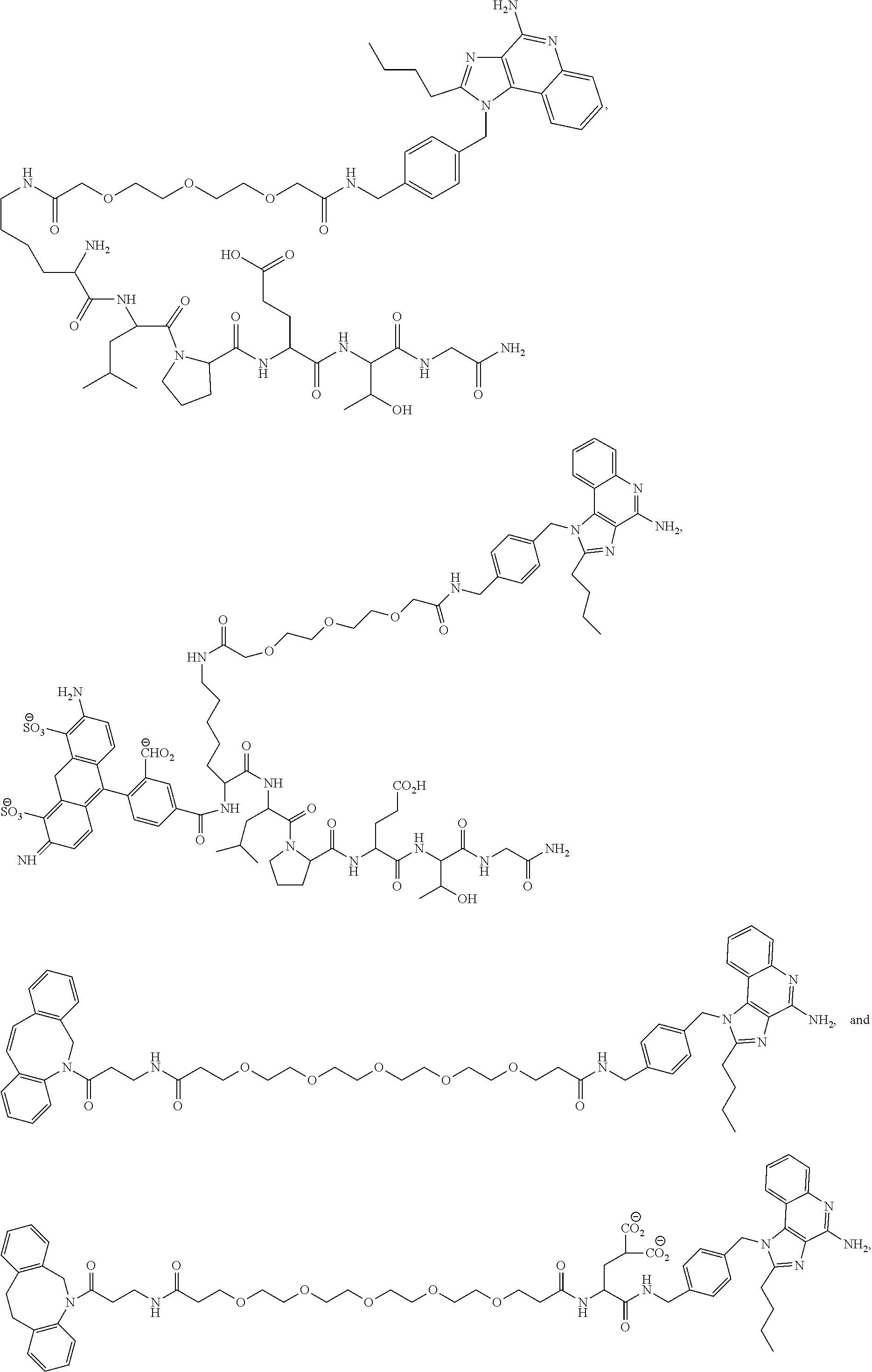
and any derivative thereof.

15. A method for treating or preventing an infection in a subject, the method comprising administering to the subject a composition comprising a cell comprising an immunomodulatory molecule bound to the cell surface.

16. The method of claim 15, wherein the infection is an infection of a pathogenic bacterium.

17. The method of claim 16, wherein the infection is a Staphylococcus aureus infection.

18. The method of claim 15, wherein an immune response is generated in the subject.

19. A method for linking a molecule to a cell surface, wherein the cell comprises a free amine on the cell surface, the method comprising:
attaching a first moiety capable of participating in a click chemistry reaction to the free amine on the surface of the cell; and
incubating the cell with a mixture comprising a molecule comprising a second moiety capable of participating in a click chemistry reaction.

20. The method of claim 19, wherein the step attaching a first moiety capable of participating in a click chemistry reaction to a free amine on the surface of the cell comprises attaching an azide to a free amine on the surface of the cell.

21. The method of claim 19, wherein the second moiety capable of participating in a click chemistry reaction is selected from the group consisting of a terminal alkyne and a cyclooctyne.

22. The method of claim 21, wherein the mixture further comprises copper.


SEQUENCE LISTING (15 sequences)

SEQ ID NO: 1 - protein, 5 aa, Artificial Sequence
  chemically synthesized
LPETG

SEQ ID NO: 2 - DNA, 24 bp, Artificial Sequence
  Chemically Synthesized
aagccagagtccttcagagagata

SEQ ID NO: 3 - DNA, 28 bp, Artificial Sequence
  Chemically Synthesized
taagggtctgcttctcccacaggaggtt

SEQ ID NO: 4 - DNA, 28 bp, Artificial Sequence
  Chemically Synthesized
cacaagaacgagagttgcctggctacta

SEQ ID NO: 5 - DNA, 28 bp, Artificial Sequence
  Chemically Synthesized
cacaagaacgagagttgcctggctacta

SEQ ID NO: 6 - DNA, 23 bp, Artificial Sequence
  Chemically Synthesized
atgagaactacagcaccagcttc

SEQ ID NO: 7 - DNA, 23 bp, Artificial Sequence
  Chemically Synthesized
gagaacctgggagtagacaaggt

SEQ ID NO: 8 - DNA, 23 bp, Artificial Sequence
  Chemically Synthesized
gagaacctgggagtagacaaggt

SEQ ID NO: 9 - DNA, 21 bp, Artificial Sequence
  Chemically Synthesized
ctgctgctggtgatcctcttg

SEQ ID NO: 10 - DNA, 24 bp, Artificial Sequence
  Chemically Synthesized
gcactgggtggaatgagactattg

SEQ ID NO: 11 - DNA, 24 bp, Artificial Sequence
  Chemically Synthesized
ttctgaggcatcaactgacaggtc

SEQ ID NO: 12 - DNA, 22 bp, Artificial Sequence
  Chemically Synthesized
cagttgtctaatgggaacgtca

SEQ ID NO: 13 - DNA, 22 bp, Artificial Sequence
  Chemically Synthesized
cagttgtctaatgggaacgtca

SEQ ID NO: 14 - DNA, 20 bp, Artificial Sequence
  Chemically Synthesized
gctgccgaagatggcggagg

SEQ ID NO: 15 - DNA, 19 bp, Artificial Sequence
  Chemically Synthesized
accaccaccttccggccca